(19) United States
(12) Patent Application Publication
(10) Pub. No.: US 2026/0058043 A1
(43) Pub. Date: Feb. 26, 2026

(54) BENDING MAGNET

(71) Applicant: Mevion Medical Systems, Inc., Littleton, MA (US)

(72) Inventors: Honghai Song, Stonybrook, NY (US); Yan Zhang, Hudson, MA (US); Xunjie Yu, Southbridge, MA (US); James Cooley, Boxborough, MA (US); Gerrit Townsend Zwart, Durham, NH (US); Mark R. Jones, Bolton, MA (US)

(73) Assignee: Mevion Medical Systems, Inc., Littleton, MA (US)

(21) Appl. No.: 19/100,037

(22) PCT Filed: Aug. 1, 2023
(86) PCT No.: PCT/US2023/029203

§ 371 Date: Jan. 30, 2025

Related U.S. Application Data
(60) Provisional application No. 63/394,461, filed on Aug. 2, 2022.

(51) Int. Cl. H01F 6/06; A61N 5/10

(52) U.S. Cl. CPC H01F 6/06; A61N 5/1081

(57) ABSTRACT

An example magnet includes an assembly. The assembly includes: (i) sets of coils for conducting current to produce a magnetic field, and (ii) a support structure on which the sets of coils are disposed asymmetrically, and a ferromagnetic yoke surrounding part of the assembly. The ferromagnetic yoke and the assembly are bent.

### CROSS-REFERENCE TO RELATED APPLICATION

This application claims priority to U.S. Provisional Application No. 63/394,461, which was filed on Aug. 2, 2022. The contents of U.S. Provisional Application No. 63/394,461 are incorporated herein by reference.

### TECHNICAL FIELD

This specification describes examples of bending magnets, such as cosine-theta magnets, that are for use in a gantry in a particle therapy system.

### BACKGROUND

Particle therapy systems use a particle accelerator to generate a particle beam for treating afflictions, such as tumors. Particle therapy systems may use a gantry to direct the particle beam toward a patient from multiple angles. In some examples, a gantry includes a device that supports a radiation delivery apparatus during treatment. The gantry includes magnetics to direct the particle beam to its destination.

### SUMMARY

An example magnet includes an assembly. The assembly includes: (i) sets of coils for conducting current to produce a magnetic field, and (ii) a support structure on which the sets of coils are disposed asymmetrically, and a ferromagnetic yoke surrounding part of the assembly. The ferromagnetic yoke and the assembly are bent. The magnet may be bent as a result. The example magnet may include one or more of the following features, either alone or in combination.

The sets of coils may include a first coil and a second coil. The first coil and the second coil may be for conducting current to produce a magnetic field. The first coil and the second coil may be disposed on the support structure asymmetrically in a first hemisphere of the magnet such that a first spacing between the first coil and the second coil in a first quadrant of the magnet may be different from a second spacing between the first coil and the second coil in a second quadrant of the magnet. The first quadrant and the second quadrant may be within the first hemisphere. The sets of coils may include a third coil and a fourth coil. The third coil and the fourth coil may be for conducting current to produce a magnetic field. The third coil and the fourth coil may be disposed on the support structure asymmetrically in a second hemisphere of the magnet such that a third spacing between the third coil and the fourth coil in a third quadrant of the magnet is different from a fourth spacing between the third coil and the fourth coil in a fourth quadrant of the magnet. The third quadrant and the fourth quadrant may be within the second hemisphere. An asymmetry of the first and second coils in the first and second quadrants, respectively, may mirror an asymmetry of the third and fourth coils in the third and fourth quadrants, respectively.

The first spacing and the third spacing may be equal. The second spacing and the fourth spacing may be equal. The first spacing and the third spacing may be less than the second spacing and the fourth spacing. The first spacing and the third spacing may be at an inner bend radius of the assembly and the second spacing and the fourth spacing may be at an outer bend radius of the assembly. In the case of an implementation containing the first through fourth coils, the ferromagnetic yoke may include notches adjacent to the assembly. The notches may be asymmetric in the first quadrant and the second quadrant. An asymmetry of the notches may be with respect to at least one of a size, shape, or placing of the notches. An asymmetry of the notches in the third and fourth quadrants, respectively, may mirror an asymmetry of the notches in the first and second quadrants, respectively.

The sets of coils may include a fifth coil and a sixth coil. The fifth coil and the sixth coil may be for conducting current to produce a magnetic field. The fifth coil may be disposed on the support structure in the first hemisphere. The sixth coil may be disposed on the support structure in the second hemisphere. A fifth spacing between the fifth coil and an adjacent one of the first or second coils in the first quadrant may be different than a sixth spacing between the fifth coil and an adjacent one of the first or second coils in the second quadrant. A seventh spacing between the sixth coil and an adjacent one of the third or fourth coils in the third quadrant may be different than a eighth spacing between the sixth coil and an adjacent one of the third or fourth coils in the fourth quadrant. An asymmetry of the first, second, and fifth coils in the first and second quadrants, respectively, may mirror an asymmetry of the third, fourth, and sixth coils in the third and fourth quadrants, respectively.

The fifth spacing and the seventh spacing may be equal. The sixth spacing and the eighth spacing may be equal. The fifth spacing and the seventh spacing may be less than the sixth spacing and the eighth spacing. The fifth spacing and the seventh spacing may be at the inner bend radius of the assembly. The sixth spacing and the eighth spacing may be at the outer bend radius of the assembly. In the case of an implementation containing the first through sixth coils, the ferromagnetic yoke may include notches adjacent to the assembly. The notches may be asymmetric in the first quadrant and the second quadrant. An asymmetry of the notches may be with respect to at least one of a size, shape, or placing of the notches. An asymmetry of the notches in the third and fourth quadrants, respectively, may mirror an asymmetry of the notches in the first and second quadrants, respectively.

The sets of coils may include a seventh coil and an eighth coil. The seventh coil and the eighth coil may be for conducting current to produce a magnetic field. The seventh coil may be disposed on the support structure in the first hemisphere. The eighth coil may be disposed on the support structure in the second hemisphere. A ninth spacing between the seventh coil and an adjacent one of the first, second, or fifth coils in the first quadrant may be different than a tenth spacing between the seventh coil and an adjacent one of the first, second, or fifth coils in the second quadrant. An eleventh spacing between the eighth coil and an adjacent one of the third, fourth, or sixth coils in the third quadrant may be different than a twelfth spacing between the eighth coil and an adjacent one of the third, fourth, or sixth coils in the fourth quadrant. An asymmetry of the first, second, fifth, and seventh coils in the first and second quadrants, respectively, may mirror an asymmetry of the third, fourth, sixth, and eighth coils in the third and fourth quadrants, respectively.

The ninth spacing and the eleventh spacing may be equal. The tenth spacing and the twelfth spacing may be equal. The ninth spacing and the eleventh spacing may be less than the tenth spacing and the twelfth spacing. The ninth spacing and the eleventh spacing may be at the inner bend radius of the assembly. The tenth spacing and the twelfth spacing may be at the outer bend radius of the assembly. In the case of an implementation containing the first through eighth coils, the ferromagnetic yoke may include notches adjacent to the assembly. The notches may be asymmetric in the first quadrant and the second quadrant. An asymmetry of the notches may be with respect to at least one of a size, shape, or placing of the notches. An asymmetry of the notches in the third and fourth quadrants, respectively, may mirror an asymmetry of the notches in the first and second quadrants, respectively.

The ferromagnetic yoke may be, or include, iron. The support structure may be non-ferromagnetic. The magnet may be bent by 60° or more relative to a straight line passing through a center of an unbent part of the magnet. The magnet may be bent by 70° or more relative to a straight line passing through a center of an unbent part of the magnet. The magnet may be bent by 80° or more relative to a straight line passing through a center of an unbent part of the magnet. The magnet may be bent by 90° or more relative to a straight line passing through a center of an unbent part of the magnet.

The magnet may be, or include, a cosine-theta magnet in which current through the sets coils has a greater concentration near a 0° or 180° location of the magnet than near a 90° or −90/270° location of the magnet. The sets of coils may be configured for dipole functionality. The sets of coils may be configured for quadrupole functionality. The sets of coils may be configured for sextupole functionality. The sets of coils may include superconducting material making the magnet superconducting. The magnet may include one or more magnetic shims that are movable relative to the ferromagnetic yoke to change a magnetic field produced by the magnet.

An example system may include a gantry that includes a beamline structure configured to direct a particle beam that is monoenergetic from an output of a particle accelerator towards an irradiation target. The beamline structure may include bending magnets to bend the particle beam along a length of the beamline structure. At least one of the bending magnets may be or include a magnet of the type described above namely, a magnet that includes an assembly comprised of: (i) sets of coils for conducting current to produce a magnetic field, and (ii) a support structure on which the sets of coils are disposed asymmetrically, and a ferromagnetic yoke surrounding part of the assembly, where the ferromagnetic yoke and the assembly are bent, and where the magnet includes one or more of the foregoing features described above.

The system may include an energy degrader that is the sole mechanism by which to actively control a change in energy of the particle beam after the particle beam is output by the particle accelerator and prior to the particle beam reaching the irradiation target. The beamline structure may be configured so as not to actively control the energy of the particle beam after the particle beam is output by the particle accelerator and prior to the particle beam reaching the energy degrader.

The at least one bending magnet may include a magnet having a magnetic field of 2.5 Tesla (T) or more. The at least one bending magnet may include a magnet having a magnetic field of 3 Tesla (T) or more. The system may include a collimator downstream of the gantry relative to the particle accelerator. The collimator may be for blocking at least part of the particle beam prior to at least part of the particle beam reaching the irradiation target. The gantry may include a support structure configured to move part of the beamline structure in a circular path around the irradiation target. The support structure may have a dimension that is 6 meters or less. The dimension may be a diameter of the support structure. A length of the beamline structure may be 6 meters (m) or less. A length of the beamline structure may be 5 meters (m) or less. An energy of the particle beam may not vary within the beamline structure by more than 1%. A distance between an output of the beamline structure and an isocenter containing the irradiation target may be 1.5 meters (m) or less.

The beamline structure may include an output channel having at least some of the bending magnets. The at least some bending magnets may include magnetic dipoles arranged in series to bend the particle beam by at least 90°. A magnetic dipole may include the at least one bending magnet. The at least one bending magnet may precede the output channel in a direction of travel of the particle beam.

The gantry may be an achromat from an entry point of a particle beam into the gantry to an isocenter of the system at which a patient is treated.

Any two or more of the features described in this specification, including in this summary section, may be combined to form implementations not specifically described in this specification.

Control of the various systems described herein, or portions thereof, may be implemented via a computer program product that includes instructions that are stored on one or more non-transitory machine-readable storage media and that are executable on one or more processing devices (e.g., microprocessor(s), application-specified integrated circuit(s), programmed logic such as field programmable gate array(s), or the like). The systems described herein, or portions thereof, may be implemented as an apparatus, method, or a medical system that may include one or more processing devices and computer memory to store executable instructions to implement control of the stated functions. The devices (e.g., magnets), systems, and/or components described herein may be configured, for example, through design, construction, composition, arrangement, placement, programming, operation, activation, deactivation, input(s), and/or control.

The details of one or more implementations are set forth in the accompanying drawings and the following description. Other features and advantages will be apparent from the description and drawings, and from the claims.

### DESCRIPTION OF THE DRAWINGS

FIG. 1 is a diagram showing a partially transparent perspective view of an example particle therapy system having an example gantry of the type described herein.

FIG. 2 is a cut-away, side view of components of the particle therapy system shown in FIG. 1, including the example gantry.

FIG. 3 is a cut-away, close-up, side view of components included in in a nozzle and an example beamline structure that may be part of the gantry shown in FIG. 1.

FIG. 4 is a cut-away, close-up, side view of components included in a nozzle and an example beamline structure that may be part of the gantry shown in FIG. 1.

FIG. 5 is an illustration of a front view of an example scanning magnet configured to scan a particle beam in two orthogonal dimensions.

FIG. 6 is a cut-away, close-up, side view of components included in a nozzle and an example beamline structure that may be part of the gantry shown in FIG. 1.

FIG. 7 is an illustration of a front view of an example scanning magnet configured to scan a particle beam in a single dimension.

FIG. 8 is an illustration of a front view of an example scanning magnet configured to scan a particle beam in a single dimension.

FIG. 9 is a cut-away, close-up, side view of components included in a nozzle and an example beamline structure that may be part of the gantry shown in FIG. 1.

FIG. 10 is a cut-away, close-up, side view of components included in a nozzle and an example beamline structure that may be part of the gantry shown in FIG. 1.

FIG. 11 is an illustration of a front view of an example superconducting scanning magnet configured to scan a particle beam in two orthogonal dimensions.

FIG. 12a is an illustration of a front view of an example superconducting scanning magnet configured to scan a particle beam in a single dimension; and FIG. 12b is an illustration of a front view of an example superconducting scanning magnet configured to scan a particle beam in a single dimension orthogonal to the dimension of FIG. 12a.

FIG. 13 is a drawing showing a perspective view of an example configurable collimator that may be part of the particle therapy system of claim 1.

FIG. 14 is a drawing showing a front, view of the configurable collimator of FIG. 13.

FIG. 15 is a drawing showing a perspective, partially-transparent view of the configurable collimator of FIGS. 13 and 14.

FIG. 16 is a block diagram of an example treatment space that is configured to house all or part of the particle therapy system of FIG. 1.

FIG. 17 is a graph showing example horizontal (x) and vertical (y) particle beam envelopes produced in the example gantry described herein.

FIG. 18 is a graph showing an example achromatic lattice design for the beamline of the example gantry described herein.

FIG. 19 is a graph showing results produced by scanning the particle beam in the horizontal (x) and vertical (y) planes using the example gantry described herein.

FIG. 20 is a cut-away, side view of components in an example particle accelerator that may be used with the particle therapy system described herein.

FIG. 21 is a perspective view of an example particle therapy system.

FIG. 22 is a perspective view of an example energy degrader.

FIG. 23 is a front, cut-away view of an example superconducting magnet that may be used as a scanning magnet in the particle therapy system of claim 1.

FIG. 24 is a cut-away view of an example superconducting coil that may be used in any superconducting magnet described herein.

FIG. 25 is a perspective view of coils an example bending magnet.

FIG. 26 is a cross-sectional view of the example bending magnet having three coils in each hemisphere.

FIG. 27 is a perspective cross-section view of the example bending magnet showing magnetic field strength in shading, with darker shades indicating greater magnetic field strength.

FIG. 28 is a perspective view of components of the example bending magnet, part of which are shown as transparent.

FIG. 29 is a perspective view of components of the example bending magnet.

FIG. 30 is a cross-sectional view of part of an example bending magnet having two coils in each hemisphere.

FIG. 31 is a cross-sectional view of part of an example bending magnet having four coils in each hemisphere.

Like reference numerals in different figures indicate like elements.

### DETAILED DESCRIPTION

Described herein are example particle therapy systems that may house a patient and an accelerator in the same space. An example system includes a particle accelerator that may be, but is not limited to, a synchrocyclotron that has low radiation leakage and that is small enough to fit within a standard linear accelerator (LINAC) vault. The system also includes a medical gantry configured to deliver a charged particle beam, such as protons or ions, output from the accelerator to treat tumors or other conditions in a patient. The gantry includes a beamline structure to direct the particle beam from the accelerator to a treatment position and to deliver the particle beam to the treatment position. The beamline structure includes magnetics, such as one or more magnetic dipoles and one or more magnetic quadrupoles, to direct the particle beam towards the treatment position. To enable delivery of the particle beam in the same space that is used for treatment, particularly in relatively small spaces such as a standard LINAC vault, at least some of the magnetics in the beamline structure are configured to bend the particle beam at angles approaching or exceeding right angles. In an example, the magnetics are configured and arranged to bend the particle beam by 70° or greater including 90° angles and obtuse angles that are greater than 90°.

The magnetics in the gantry may include one or more magnets, such as a cosine-theta bending magnet having the following features that enable the particle beam to be bent magnetically: sets of current-conducting coils (or simply “coils”) including at least first and second coils, where the first and second coils are for conducting current to produce a magnetic field, and a non-ferromagnetic support structure (“support”) on which the sets of coils are arranged asymmetrically. An assembly comprised of the coils and the support encloses, at least in part, an air core through which a particle beam passes. A ferromagnetic brick or yoke surrounds, at least in part, the assembly and the air core, but is otherwise solid except, possibly, for notches in the yoke that are adjacent to an external part of the assembly. The notches define channels through the yoke, which may be filled with air or vacuum. The notches affect the amount of ferromagnetic material adjacent to the coils and, thus, affect the shape the magnetic field produced by the coils. The cosine-theta bending magnet is bent or curved. The configuration of the magnet—for example, the asymmetric coil windings and the asymmetric notches in the yoke—enables the magnet to reduce particle beam distortion during its travel through the gantry, particularly at bends in the gantry. For example, the cross-sectional (e.g., circular) shape of the particle beam may be maintained substantially or wholly circular during travel through the gantry.

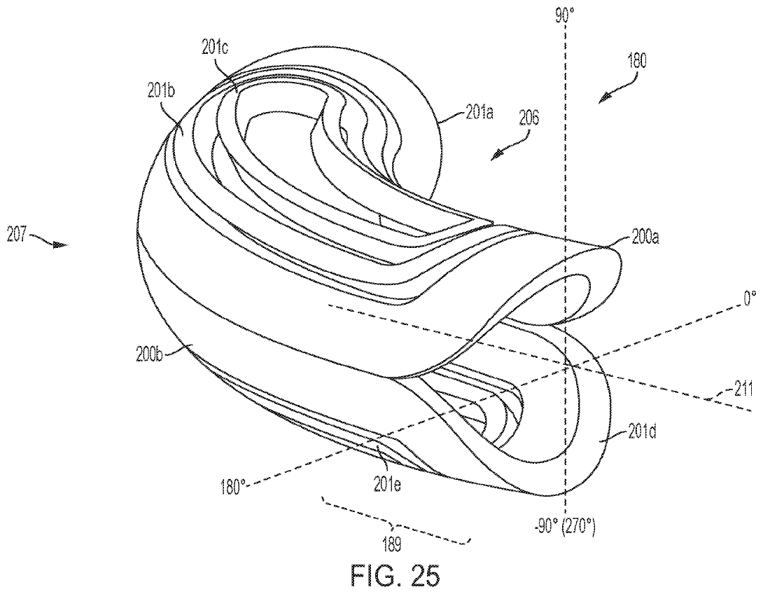

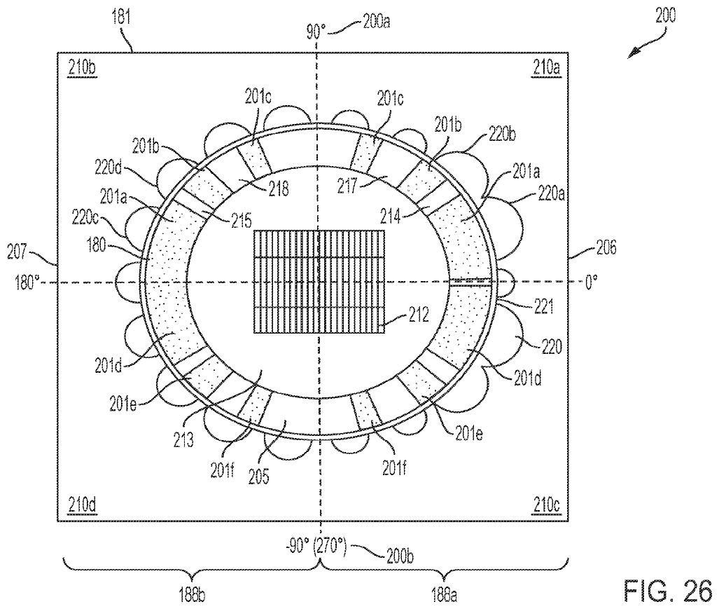

FIG. 25 shows a perspective view of example coils 180 for an example cosine-theta bending magnet (“magnet”), which may be used as a bending magnet in an example particle therapy system gantry such as those described herein. However, the magnet is not limited to use in the context. FIG. 26 shows a cross-sectional front view of an example magnet 200 that includes coils 180, a non-ferromagnetic support 205, an air core 213, and a yoke 181 comprised of iron or other ferromagnetic material.

Magnet 200 may be a dipole magnet, a quadrupole magnet, or a sextupole magnet. A dipole magnet has two poles, one north and one south. Its magnetic field lines form closed loops that emerge from the north pole, re-enter at the south pole, then pass through the body of the magnet. A quadrupole magnet includes a group of four magnetic poles laid out so that in a planar multipole expansion of the magnetic field, the dipole terms cancel and the lowest significant terms in the field equations are quadrupole. Sextupole magnets include six magnetic poles set out in an arrangement of alternating north and south poles arranged around an axis. The coils described herein may be layered in order to generate higher order field harmonics.

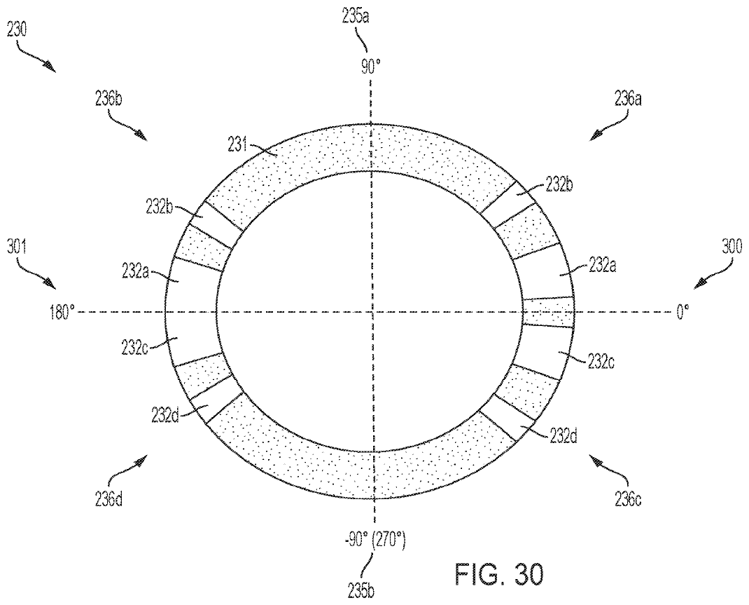

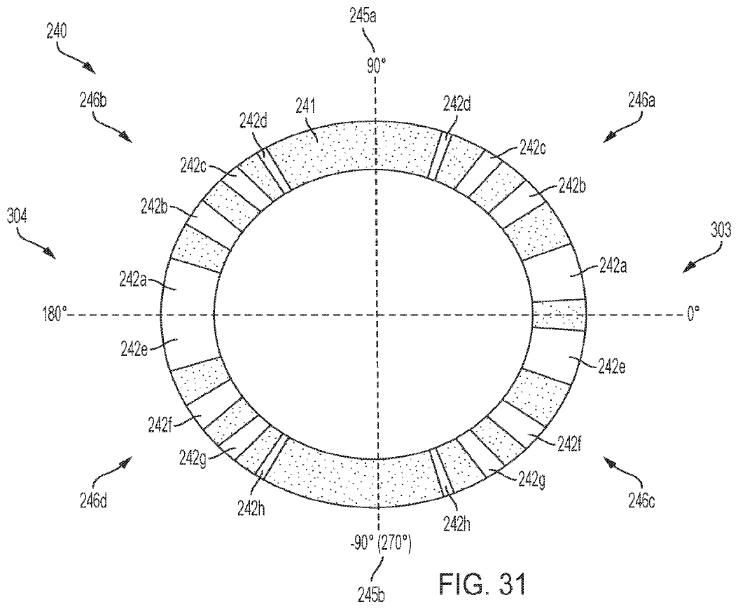

Referring to FIGS. 25 and 26, magnet 200 is an electromagnet that includes multiple current-conducting coils 180 in each of its hemispheres 200a and 200b where, in FIGS. 25 and 26, 0°to 180°corresponds to upper hemisphere 200a and 180° to 360° (0° again) corresponds to lower hemisphere. In the example of FIGS. 25 and 26, coils 180 of magnet 200 includes three coils 201a, 201b, 201c in upper hemisphere 200a and three coils 201d, 201e, 201f in its lower hemisphere 200b (coil 201f is not visible in FIG. 25). In some implementations, magnet 200 may include fewer than three coils or more than three coils in each of its hemispheres. For example, a bending magnet of the type described herein may include two coils in each hemisphere (e.g., FIG. 30), three coils in each hemisphere (e.g., FIGS. 25 to 29), four coils in each hemisphere (e.g., FIG. 31), five coils in each hemisphere, six coils in each hemisphere, and so forth. Any appropriate number of coils may be included in each hemisphere of the bending magnet.

Referring to FIG. 26, in magnet 200, lower hemisphere 200b is a mirror image of upper hemisphere 200a, meaning that the structures and relative spacings of the coils in each hemisphere are the same. For example, coils 201a and 201d have the same structure and spacing relative to coils 201b and 201e, respectively. Coils 201b and 201e have the same structure and spacing relative to coils 201c and 201f, respectively. Coils 201c and 201f have the same structure and spacing relative to the 90° and −90° (270°) locations on the magnet, respectively. Accordingly, descriptions herein of the upper hemisphere apply to the lower hemisphere and vice versa.

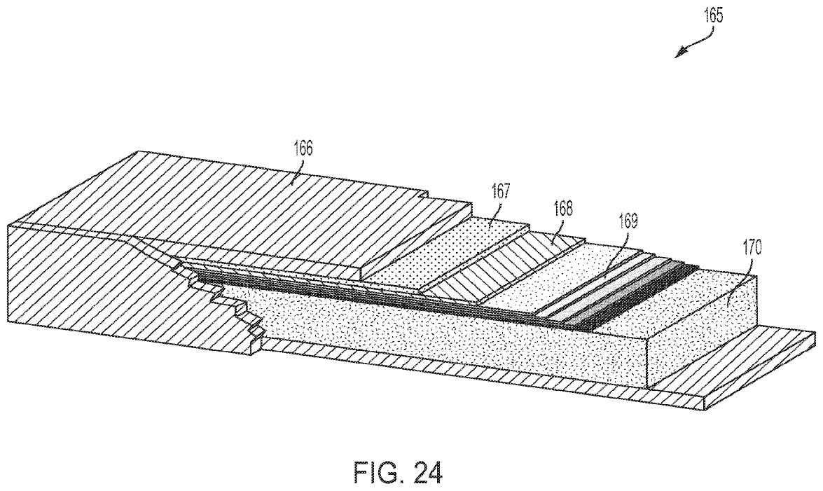

Bending magnet coils, such as coils 180, may be superconducting or non-superconducting. For example, one or more or all of the coils may be made of copper or any other appropriate non-superconducting material(s), examples of which are described herein. One or more or all of the coils may be made of superconducting material(s), examples of which are described herein. One or more or all of the coils may have a configuration as described with respect to FIG. 24 below. Due to compactness and space limitations, ends of the coils have been configured to meet predefined critical strain/stress limits and a predefined total field integral (in a non-limiting example, 3.54 Tesla-meter) on the coil mandrel(s).

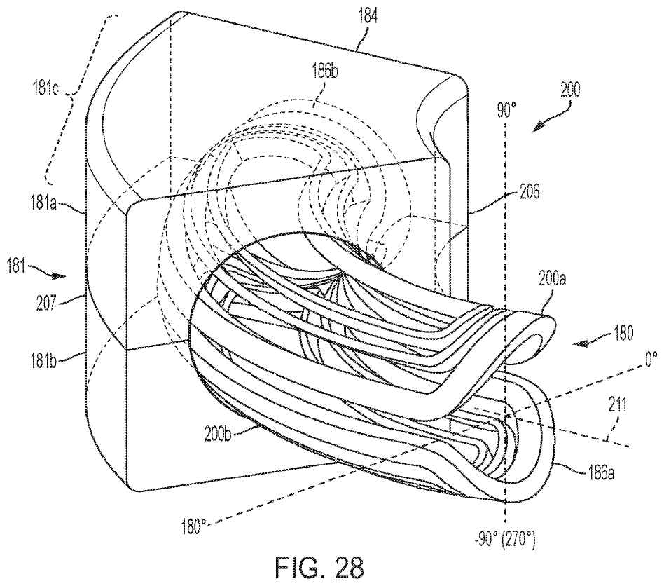

The coils are disposed (e.g., wrapped around, held, placed, arranged, or maintained) on non-ferromagnetic support 205 (FIG. 26, not shown in FIGS. 25, 28, or 29). Support 205 may be made of, or include, a non-ferromagnetic material such as aluminum or stainless steel. Support 205 may be a single contiguous or integrated structure or support 205 may include multiple separate structures that, taken together, constitute the support structure. Support structure 205 may have a shape that is complementary to the shape of the coils. For example, as shown in FIG. 26, support 205 has a shape that is complementary to the shape of coils 201a, 201b, 201c, 201d, 201e, and 201f. Together, support 205 and coils 201a, 201b, 201c, 201d, 201e, and 201f define a substantially circular cross-section, as shown in FIG. 26 that defines a space that includes air core 213. Air core 213 may contain a gas such as air or a noble gas or it may approach vacuum, e.g., 10⁻⁵ Torr (0.0013332 Pascal) or less.

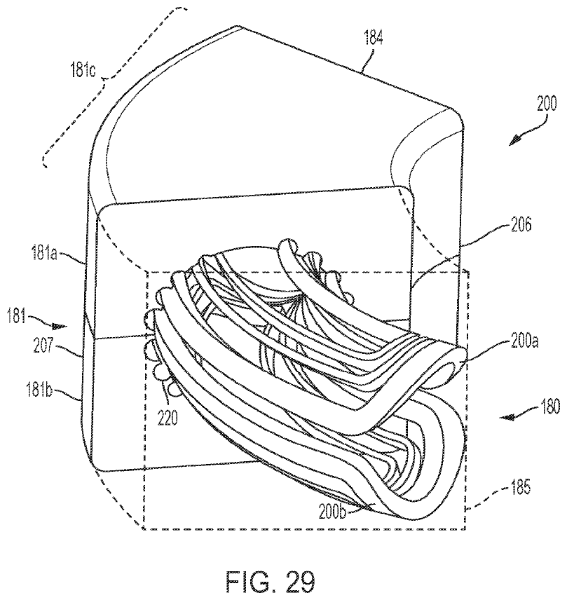

Current carried through the coils 201a, 201b, 201c, 201d, 201e, and 201f generates a magnetic field that is shaped, at least in part, by ferromagnetic yoke 181. Of course, the magnitude of the current also shapes the magnetic field. Yoke 181 may be a solid structure made of ferromagnetic material such as iron, as shown in FIGS. 28 and 29. As shown in FIGS. 28 and 29, yoke 181 may be formed from a top piece 181a and a bottom piece 181b; however, in other implementations yoke 181 may be formed of left and right pieces or more than two pieces. In FIG. 28, part 181c of yoke 181 is illustrated as transparent, whereas in FIG. 29, part 181a yoke 181 depicted in solid form. The transparent form is for the sake of showing coils 180 through the yoke and not to indicate that all or part of yoke 181 is actually transparent.

Yoke 181 surrounds, at least in part, the assembly comprised of support 205, coils 201a, 201b, 201c, 201d, 201e, 201f, and core 205. In in the example implementations of FIGS. 28 and 29, yoke 181 is shown in cut-away form to illustrate coils 180. However, in the example implementations of FIGS. 28 and 29, yoke 181 extends to cover the entire length of coils 180, from end 184 (best visible in FIG. 28) to 185 (FIG. 29, not shown in FIG. 28). The ends 186a, 186b (FIG. 28) of coil 180 remain exposed to enable connection of magnet 200 to other magnetics in a beamline and to allow a particle beam to pass through magnet 200 in the manner described herein.

In some implementations, yoke 181 includes rounded notches or channels adjacent, and around the outer surface of, coil 181. The notches may run along the entire length of yoke 181/magnet 200 and may have the same cross-section along the entire length of yoke 181/magnet 200 or their cross-sections may change along the length of yoke 181/magnet 200. For example, as shown in FIGS. 26 and 29 (not shown in FIG. 28), notches 220 are rounded or semi-circular and extend around the circular cross-section of the assembly comprised of support 205, coils 201a, 201b, 201c, 201d, 201e, 201f, and core 213. Lower hemisphere 200b is a mirror image of upper hemisphere 200a such that the notches on the lower hemisphere 200b are a mirror image of the notches on upper hemisphere 200a. By contrast, notches 220 on right hemisphere 188a and left hemisphere 188b are asymmetric to account for the bend in magnet 200. In an example, the asymmetry is that the notches, on average, have greater volume/size in right hemisphere 188a than in left hemisphere 188b, resulting in more ferromagnetic material being present in left hemisphere 188b than in right hemisphere 188a. In an example, the asymmetry is that the notches are differently shaped in right hemisphere 188a than in left hemisphere 188b causing more ferromagnetic material to be present in left hemisphere 188b than in right hemisphere 188a. In an example, the asymmetry is that the notches, on average, are closer together in right hemisphere 188a than in left hemisphere 188b, resulting in more ferromagnetic material being present in left hemisphere 188b than in right hemisphere 188a. In an example, the asymmetry is that the notches, on average, are both closer together and larger on average in right hemisphere 188a than in left hemisphere 188b, resulting in more ferromagnetic material being present in left hemisphere 188b than in right hemisphere 188a. For example, in FIG. 26, notches 220a and 220b in right hemisphere 188a are larger and closer together (in fact, they overlap) than their counterpart notches 220c and 220d in left hemisphere 188b. In an example, the asymmetry is that the number of notches in right hemisphere 188a is greater than the number of notches left hemisphere 188b, resulting in more ferromagnetic material being present in left hemisphere 188b than in right hemisphere 188a. Any asymmetry resulting from notch configuration, placement, number, size, shape, and/or other factors may be used to shape the magnetic field. The placement and configuration of the notches affects the magnetic field to enable transmission, and maintain integrity of, the particle beam.

Referring to FIGS. 25 and 26, in magnet 200, there is more current-conducting coil closer to the 0°/180° location than there is at the 90°. Accordingly, while the magnet is operational—that is, while the current is conducting through the coils—there is greater current density closer to 0°/180° than there is at 90°. In the example presented, there are no conductors at 90°; therefore, current density at 90° is zero. As noted, in this example, lower hemisphere 200b is a mirror image of upper hemisphere 200a. Accordingly, in lower hemisphere 200b, while the current is conducting through the coils, there is greater current density closer to 0°/180° than there is at −90°/270°.

Another feature of magnet 200 is that the coils 201a, 201d closer to 0°/180° have greater current-carrying capacity than the coil 201c closer to 90° and coil 201f closer to −90°/270°. For example, coils 201a, 201d each has a greater cross-sectional area than coils 201c, 201f, respectively. Generally, in implementations of magnet 200, in each quadrant 210a, 210b, 210c, and 210d (FIG. 26), the current-carrying capacity of the coils decreases from 0°/180° to 90° and 0°/180° to −90°/270°.

In this regard, quadrant 210a extends from 0° to 90°; quadrant 210b extends from 90° to 180°; quadrant 210d extends from 180° to −90°/270°; and quadrant 210c extends from 270° to 360°/0°. In this example, coil 201b, which is between coils 201a and 201c has a cross-sectional area that is less than the cross-sectional area of coil 201a and greater than the cross-sectional area of coil 201c. Likewise, coil 201e, which is between coils 201d and 201f has a cross-sectional area that is less than the cross-sectional area of coil 201d and greater than the cross-sectional area of coil 201f. The reduction in current-carrying capacity from 0°/180° to 90° and 0°/180° to −90°/270° may be constant or vary. In the constant example, for hemisphere 200a, coil 201c may have 20% less current-carrying capacity than coil 201b; and for hemisphere 200a, coil 201b may have 20% less current-carrying capacity than coil 201a. In the varying example, coil 201c may have 20% less current-carrying capacity than coil 201b; and coil 201b may have 10% less current-carrying capacity than coil 201a. The same differences in current-carrying capacity may hold for the coils counterparts in hemisphere 200b. The 10% and 20% numbers are 201 non-limiting examples; and the reduction in current-carrying capacity from 0°/180° to 90° (or 0°/180° to −90°/270°) in the coils of the cosine-theta magnet herein may be greater or less than these numbers.

As shown in FIG. 25 and 27 to 29, cosine-theta magnet 200 bends. This feature is particularly useful for directing a particle beam in a compact gantry of the types described herein. The cosine-theta magnet may bend, and therefore bend the particle beam, by 10° or more, 20° or more, 30° or more, 40° or more, 50° or more, 60° or more, 70° or more, 80° or more, 90° or more, 100° or more, 110° or more, 120° or more, 130° or more, 140° or more, 150° or more, 160° or more, 170° or more, or to 180° relative to a straight line 211 (FIGS. 25 and 28), where the straight line 211 passes through, and follows, the track of, an unbent part of magnet 200. Magnet 200 may bend at any appropriate angle. The bending may be any degree with a range of 0° to 90°, any degree within a range of 0° to 180°, or any degree within a range of 70° to 180° relative to straight line 211.

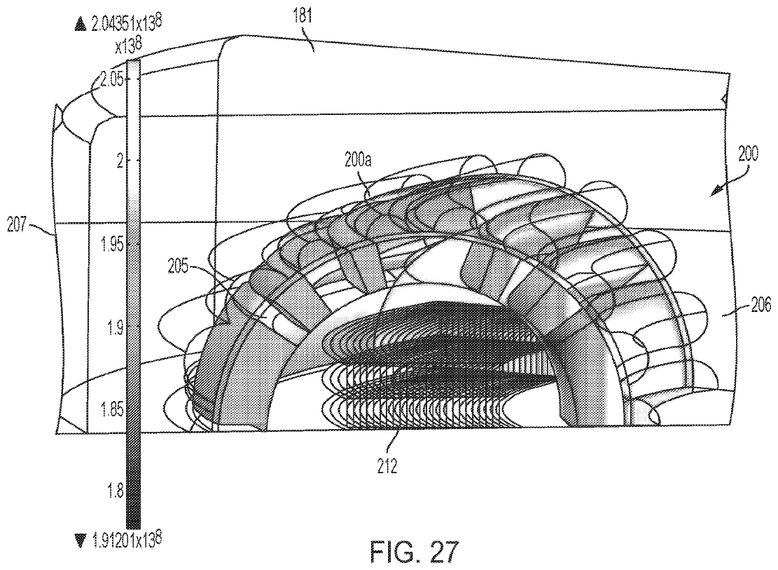

The homogeneity of the magnetic field in the rectangular region 212 (FIGS. 26, 27) of core 213 of magnet 200, where the particle beam is constrained to travel by the magnetic field, may be distorted by the bend in the cosine-theta magnet. More specifically, the bend in the magnet may cause the cross-section of the particle beam—that is, the spots—to become elliptical instead of remaining circular. For example, the spot size (e.g., cross-sectional area) of the particle beam may have an aspect ratio of 5% or more and may grow in size from 3 millimeters (mm) or 4 mm sigma to 10 mm sigma in one or more planes due to the bending. Magnet 200; however, is configured to counteract such distortion and to maintain the cross-section of the particle beam in region 212 at a predefined shape, such as a circle. To counteract such distortion at least in part, the sets of coils in magnet 200 are disposed asymmetrically on support 205 in each hemisphere 200a, 200b to shape the magnetic field so as to prevent or reduce distortion. For example, as shown in FIG. 26, in hemisphere 200a, the spacing 214 between coils 201a and 201b in quadrant 210a is different than the spacing 215 between coil 201a and 201b in quadrant 210b. Likewise, in the same hemisphere 200a, the spacing 217 between coils 210b and 210c in quadrant 210a is different than the spacing 218 between coils 210b and 210c in quadrant 210b. The same spacing differences between coils in hemisphere 200 are present in the mirror-image coils in hemisphere 200b. That is, the difference in spacing between coils 201d, 201e and 201e, 201f is the same as the difference in spacing, respectively, between coils 201a, 201b and 201b, 201c, respectively. In some implementations, the difference in coil spacing in different quadrants may be different in different hemispheres, In this example, the spacings between pairs of coils in quadrant 210a is greater than the spacing between the same pairs of coils in quadrant 201b; however, in other implementations, the spacing between pairs of coils in quadrant 210s may be less than the spacing between the same pairs of coils in quadrant 201b As shown in FIGS. 25 to 29, in magnet 200, the curvature or bend in magnet 200 produces inner surface 206 and outer surface 207, with the inner surface having a smaller radius of curvature or bend radius than the outer surface. The inner surface and outer surface may refer to the coils, the assembly, or to the magnet, since the bend radius is the same or substantially the same. In this example, coils 201a, 201b, 201c, 201d, 201e, and 201f are spaced such that the spacing between the coils is less on the outer surface 207 than on the inner surface 206. For example, at outer surface 207 and quadrant 210b of magnet 200, coils 201a, 201b, and 201c are closer together they are at inner surface 206 and in quadrant 210a of magnet. In this configuration, for example, space 215 is smaller than space 214 and space 218 is smaller than space 217. The differences in spacing may be on the order of 1%, 2%, 3%, 4%, 5%, 6%, 7%, 8%, 9%, or more. For example, space 215 may be 1%, 2%, 3%, 4%, 5%, 6%, 7%, 8%, 9%, or more smaller than space 214; and space 218 may be 1%, 2%, 3%, 4%, 5%, 6%, 7%, 8%, 9%, or more smaller than space 217. Any appropriate spacing may be used to achieve the effects described herein.

These spacing differences may be mirrored in the coils located in lower hemisphere 200b. More specifically, coils 201d, 201e, and 201f, since they are arranged in a mirror-image configuration of coils 201a, 201b, and 201c, will have the same spacing variations as coils 201a, 201b, and 201c except they will be in quadrant 201d, which will have the same coil spacing as quadrant 201b, and in quadrant 201c, which will have the same coil spacing as quadrant 201a.

The combination of the coil 180 asymmetry in the right hemisphere 188a and left hemisphere 188b, the greater current-carrying capacity of coils closer to 0° (e.g., 201a, 201d) than to 90° (e.g., 201c, 201f), and notch 220 asymmetry in the right hemisphere 188a and left hemisphere 188b shapes the magnetic field of magnet 220 at region 212 so as to (i) keep the particle beam within region 212, and (ii) to keep the particle beam circular or substantially circular, as the particle beam travels the length of magnet 200 including through the part of magnet 200 that bends. In some examples, substantially circular may include a 9% or less, 8% or less, 7% or less, 6% or less, 5% or less, 4% or less, 3% or less, 2% or less, or 1% or less deviation from perfectly circular. Other factors that affect the magnitude and shape of the magnetic field include, but are not limited to, the current-carrying capacity of the coils, the size and shape of yoke 181, and the materials from which the various components of magnet 200 are made.

FIG. 27 shows the magnitude and locations of the magnetic field 206 in greyscale or color for upper hemisphere 200a for the example implementation of FIGS. 25, 26, 28, and 29. As shown, the magnetic field strength is greater in the inner surface 206 of magnet 200 than on the outer surface 207 of the magnet. The magnetic field is greater at the inner radius as a consequence of the Biot-Savart law solution for this curved conductor geometry. In this regard, if the magnetic field were the same strength across all radii (e.g., from the inner surface 206 to the outer surface 207), there would be a net focusing of the beam in the bending plane for different particle trajectories through the magnet. This can overfocus the particle beam and may be compensated by reductions in the strength of the magnetic field on the outside radius (207) relative to the strength of the magnetic field on the inside radius (206). As a result of this difference in the magnetic field, distortion of the particle beam spots—that is, the cross-section of the particle beam—may be reduced or eliminated. As a result, the particle beam may remain substantially circular as is travels through the entire length of magnet 200.

In a non-limiting example, magnet 200 achieves 0.1% homogeneity of the magnetic (e.g., dipole) field in region 212, which may be 100 millimeters (mm) by 90 mm along the beam trajectory throughout the length of the magnet.

In some implementations, one or more magnetic shims (not shown) may be used to change the amount of ferromagnetic material in yoke 181 and, thus, in the magnet. For example, the shims may be rods, cones, or other structures that are controllable to move into or out of yoke 181 to adjust the amount of ferromagnetic material in yoke 181 and, thereby, change the shape of the magnetic field produced by the magnet. In some implementations, the shims are controllable manually. In some implementations, the shims are computer controlled. For example, each shim may be connected to a computer-controlled actuator that controls movement of the shim into, or out of, the ferromagnetic core. The shims may be moved, either through manual or computer control, to be completely embedded in the yoke to be completely out of the yoke. There may be one shims per magnet quadrant—for example, two, three, four, five, and so forth—per quadrant. In an example, one or more magnetic field sensors may detect the magnetic field produced by magnet 200 and the shims may be controlled to change the magnetic field to a magnetic field having a target shape. In an example, one or more sensors may detect the location of the particle beam in core 213 and the shims may be controlled to change the magnetic field to control particle beam placement.

FIG. 30 shows an example assembly 230 that may be used in a magnet like magnet 200 (see, e.g., FIG. 26). Assembly 230 includes a support 231, which has the same function as, and may have a similar structure and composition as, support 205. Assembly 230 also includes coils 232a, 232b, 232c, and 232d. The sets of coils are disposed asymmetrically on support 231 in each hemisphere 235a, 235b to shape the magnetic field so as to prevent or reduce particle beam distortion at least in part, as described herein. For example, as shown in FIG. 30, in hemisphere 235a, the spacing between coils 232a and 232b in quadrant 236a at the magnet's inner radius of curvature 300 is different from (for example, greater than) the spacing between coils 232a and 232b in quadrant 236b at the magnet's outer radius of curvature 301. The same spacing differences between coils in hemisphere 235a are present in the mirror-image coils in hemisphere 235b. That is, in hemisphere 235b, the spacing between coils 232c and 232d in quadrant 236c is different from (for example, greater than) the spacing between coils 232c and 232d in quadrant 236 db. Assembly 230 differs from that of FIG. 26 in that assembly 230 includes two sets of coils instead of three sets of coils. Otherwise, assembly 230 may be incorporated into a magnet structure such as that shown in FIGS. 28 and 29 with all the accompanying features configured for a two-coil, rather than three-coil design. Those features include, but are not limited to, asymmetric notches and a magnetic yoke configured for a two-coil design.

FIG. 31 shows another example assembly 240 that may be used in a magnet like magnet 200 (see, e.g., FIG. 26). Assembly 240 includes a support 241, which has the same function as, and may have a similar structure and composition as, support 205. Assembly 240 also includes coils 242a, 242b, 242c, 242d, 242e, 242f, 242g, and 242h. The sets of coils are disposed asymmetrically on support 241 in each hemisphere 245a, 245b to shape the magnetic field so as to prevent or reduce particle beam distortion at least in part, as described herein. For example, as shown in FIG. 31, in hemisphere 245a, the spacing between coils 242a and 242b in quadrant 246a at the magnet's inner radius of curvature 303 is different from (for example, greater than) the spacing between coils 242a and 242b in quadrant 246b at the magnet's outer radius of curvature 304; the spacing between coils 242b and 242c in quadrant 246a is different from (for example, greater than) the spacing between coils 242a and 242b in quadrant 246b; and the spacing between coils 242c and 242d in quadrant 246a is different from (for example, greater than) the spacing between coils 242c and 242d in quadrant 246b. The same spacing differences between coils in hemisphere 245a are present in the mirror-image coils in hemisphere 245b. That is, in hemisphere 245b, the spacing between coils 242e and 242f in quadrant 246c at the magnet's inner radius of curvature is different from (for example, greater than) the spacing between coils 242e and 242f in quadrant 246d at the magnet's outer radius of curvature; the spacing between coils 242f and 242g in quadrant 246c is different from (for example, greater than) the spacing between coils 242f and 242g in quadrant 246d; and the spacing between coils 242g and 242h in quadrant 246c is different from (for example, greater than) the spacing between coils 242g and 242h in quadrant 246d. Assembly 240 differs from that of FIG. 26 in that assembly 240 includes four sets of coils instead of three sets of coils. Otherwise, assembly 240 may be incorporated into a magnet structure such as that shown in FIGS. 28 and 29 with all the accompanying features configured for a four-coil, rather than three-coil design. Those features include, but are not limited to, asymmetric notches and a magnetic yoke configured for a four-coil design.

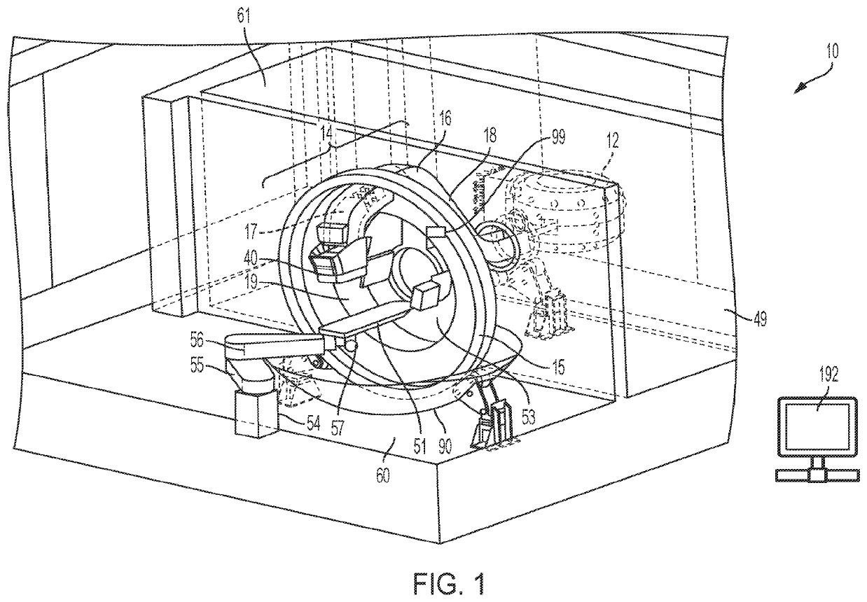

In the following description of a particle beam gantry, cosine-theta bending magnets of the type described with respect to FIGS. 25 to 31 or any variant thereof may implement any or all of the bending magnets and variants thereof described, e.g., with respect to FIGS. 1, 2, 3, 4, 6, 9, 10, 16, and 21. For example, cosine-theta magnets of the type described herein having dipole, quadrupole, and/or sextupole configurations may be used to implement the bending magnets in any of the particle beam gantries, or variants thereof, described herein.

FIG. 1 shows an example implementation of a particle therapy system 10 of the type described above that may include one or more bending magnets of the type described with respect to FIGS. 25 to 31 or variants thereof. Particle therapy system 10 includes a particle accelerator 12, examples of which are described herein. In this example, particle accelerator 12 is a synchrocyclotron having a superconducting electromagnetic structure that generates a maximum magnet field strength of 2.5 Tesla (T) or more or 3T or more. In this regard, a superconductor is an element or metallic alloy such as niobium-tin (Nb₃Sn) which, when cooled below a threshold temperature, loses most, if not all, electrical resistance. As a result, current flows through the superconductor substantially unimpeded. Superconducting coils, therefore, are capable of conducting larger currents in their superconducting state than ordinary wires of the same size. Because of the high amounts of current that they are capable of conducting, superconducting coils are particularly useful in particle therapy applications.

An example synchrocyclotron is configured to output protons or ions as a monoenergetic particle beam having an energy level of 150 MegaElectronvolts (MeV) or more. The example synchrocyclotron has a volume of 4.5 cubic meters (m³) or less and a weight of 30 Tons (T) or less. Due to its size, this type of particle accelerator is referred to as “compact”. However, as described herein, synchrocyclotrons or other types of particle accelerators having weights, dimensions, magnetic fields, and/or energy levels other than these may be used in particle therapy system 10.

Particle therapy system 10 also includes gantry 14. Gantry 14 includes a ring-shaped or circular support structure 15 and a beamline structure 16. The combination of support structure 15 and beamline structure 16 may be referred to as a “compact gantry” due to its relatively small size. Beamline structure 16 includes an output channel 17 that mounts to support structure 15 and a conduit 18 that directs the particle beam to the output channel. Gantry 14 also includes one or more motors (not shown) for moving output channel 17 around support structure 15 relative to a treatment position 19. The treatment position may include a system isocenter where a patient may be positioned for treatment. In an example, the motors may move output channel 17 along a track on structure 15 resulting in rotation of output channel 17 relative to treatment position 19. In an example, a structure to which output channel 17 is attached may rotate relative to treatment position 19, resulting in rotation of output channel 17 relative to the treatment position. In some implementations, the rotation enabled by gantry 14 allows output channel 17 to be positioned at any angle relative to the treatment position. For example, output channel 17 may rotate through 360° and, as such, output channel 17 may be positioned at 0°, 90°, 270°, and back to 0°/360° or any angle among these rotational positions.

As noted previously, beamline structure 16 is configured to direct a particle beam from accelerator 12 to treatment position 19. To this end, output channel 17 includes magnetics to bend the particle beam towards the treatment position. As noted, beamline structure 16 includes conduit 18 containing magnetics along the beamline that direct the particle beam from particle accelerator 12 to output channel 17.

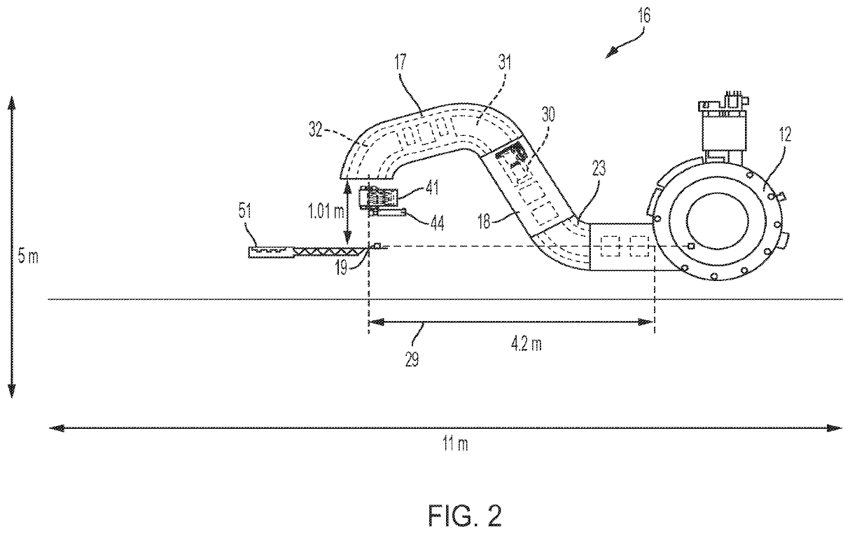

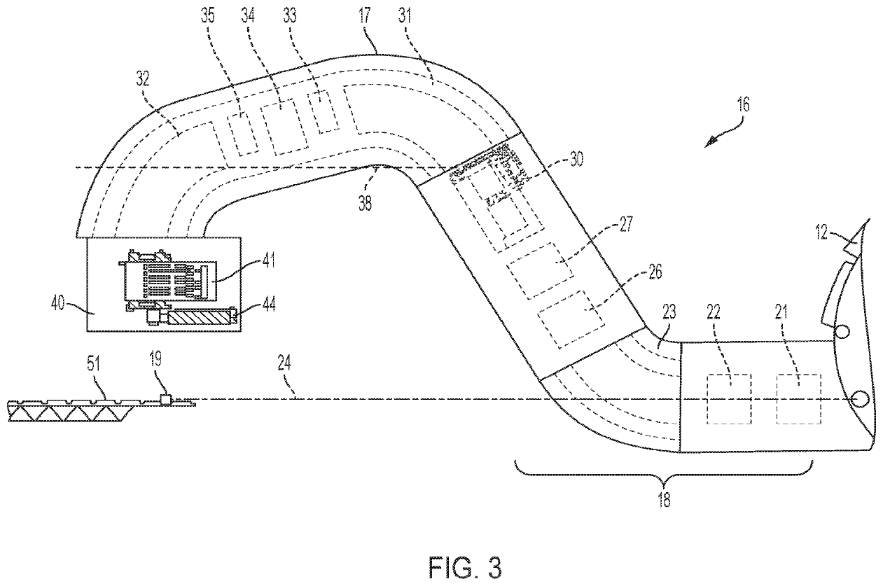

Referring to FIGS. 2 and 3, conduit 18 of example beamline structure 16 includes non-superconducting magnetic quadrupoles 21 and 22 and bending magnet 23, which may be a superconducting dipole magnet. Magnetic quadrupoles 21 and 22 are configured to keep the particle beam focused and traveling straight or substantially straight—for example, a 5% or less deviation from straight—within beamline structure 16. Magnetic quadrupoles 21 and 22 are configured to focus the particle beam to maintain a substantially consistent cross-sectional area of the particle beam, for example, to within a tolerance of ±5%. Bending magnet 23 is configured to bend the particle beam towards output channel 17, as shown in the figures. Bending magnet 23 may be configured to bend the particle beam anywhere in a range of 20° to 80° relative to horizontal 24. Generally, greater bend angles may reduce the distance between particle accelerator 12 and treatment position 19 or system isocenter, thereby reducing the space required to accommodate the gantry and, thus, the size of the particle therapy system. For example, replacing a non-superconducting bending magnet 23 with a superconducting bending magnet 23 that bends the particle beam by more than 80°—for example, by 90° or more—may further reduce the distance from particle accelerator 12 to support structure 15 and, thus, to treatment position 19 and the isocenter. Bending magnet 23 may be or include a bending magnet of the type shown in, and described with respect to, FIGS. 25 to 31 or a variant thereof.

In some implementations, higher-order magnetics may be used in place of, or in addition to, any magnetic quadrupoles described herein. For example, the beamline structure may include one or more magnetic sextupoles in place of, or in addition to, the magnetic quadrupoles. The magnetic sextupoles may be configured to keep the particle beam focused and traveling straight or substantially straight—for example, a 5% or less deviation from straight—within beamline structure 16. The magnetic sextupoles may also configured to maintain a consistent cross-sectional area of the particle beam, for example, to within a tolerance of ±5%. Also, sextupole magnets may correct for chromatic effect of a quadrupole magnet. Compared with a magnetic quadrupole, a magnetic sextupole has a greater focusing effect for particles that are displaced farther from an axis that defines an ideal location of the beamline, such as within region 212 of FIG. 26.

Referring back to FIG. 3, in this example, conduit 18 of beamline structure 16 also includes two non-superconducting magnetic quadrupoles 26 and 27. Magnetic quadrupoles 26 and 27 are configured to keep the particle beam focused and traveling straight or substantially straight—for example, a 5% or less deviation from straight—within beamline structure 16. Magnetic quadrupoles 26 and 27 are configured to maintain a consistent cross-sectional area of the particle beam, for example, to within a tolerance of ±5%. As described previously, higher-order magnetics may be substituted for one or more of the magnetic quadrupoles to improve focusing.

Particle therapy system 10 also includes one or more scanning magnets 30 in the path of the particle beam and configured to move the particle beam across at least part of a beam field that covers all or part of (that is, at least part of) the irradiation target. In some examples, the beam field includes the maximum (e.g., planar) extent that the particle beam can be moved across a plane parallel to a treatment area on a patient for a given position of the compact gantry. Movement of the particle beam across the beam field results in movement across at least part of an irradiation target at a treatment position 19. The scanning magnets may be sized and configured to move the particle beam across a beam field having an area of 20 centimeters (cm) by 20 cm or greater, although system 10 is not limited to any particular beam field size. For example, the scanning magnets may have an aperture of 20 cm by 20 cm or greater, although the scanning magnets are not limited to any particular aperture size.

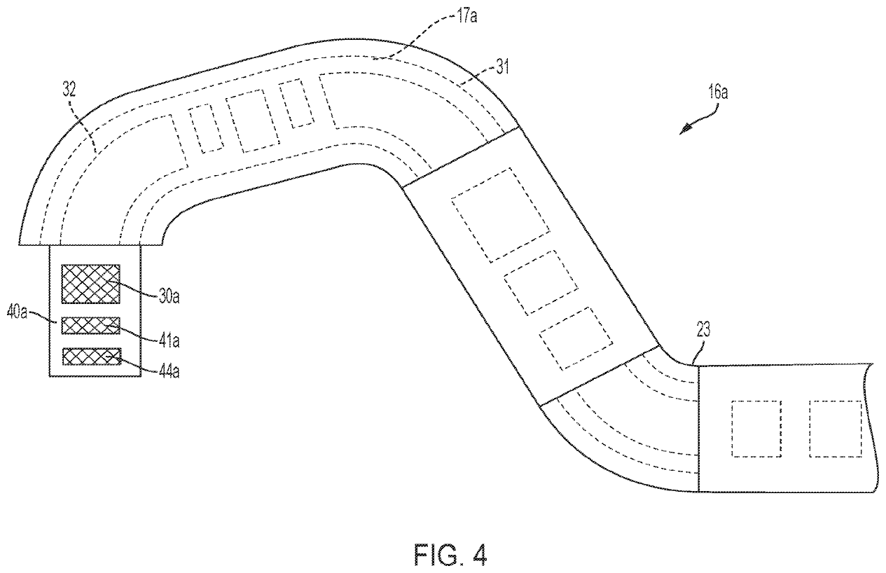

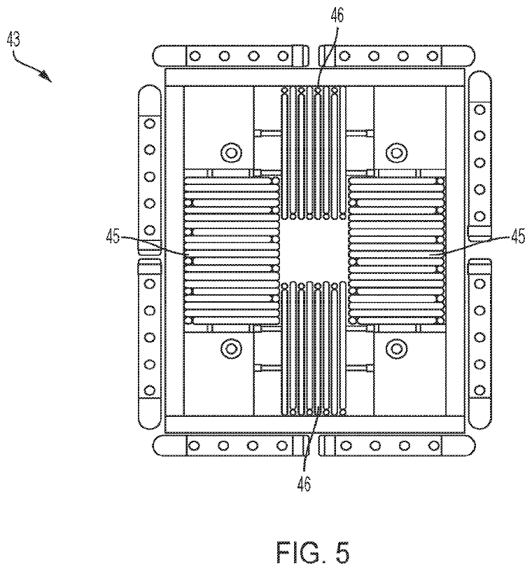

The scanning magnets may be located at different positions within the particle therapy system. For example, in beamline structure 16a shown in FIG. 4, which is a variant of beamline structure 16, all of the scanning magnets 30a may be located in nozzle 40a, along with energy degrader 41a and collimator 44a (both described below), on a path of the particle beam between output channel 17a and the treatment position. Referring to FIG. 5, an example scanning magnet 43 is controllable in two dimensions (e.g., Cartesian XY dimensions) to position the particle beam in those two dimensions and to move the particle beam across at least a part of an irradiation target. In this example, scanning magnet 43 includes a first set 45 of two coils, which control particle beam movement in the Cartesian X dimension of a defined coordinate system, and a second set 46 of two coils, which are orthogonal to the first set of two coils and which control particle beam movement in the Cartesian Y dimension. Control over movement of the particle beam may be achieved by varying current through one or both sets of coils to thereby vary the magnetic field(s) produced thereby. By varying the magnetic field(s) appropriately, the magnetic fields acts on the particle beam to move the particle beam in the X and/or Y dimension across a beam field and, thus, the irradiation target.

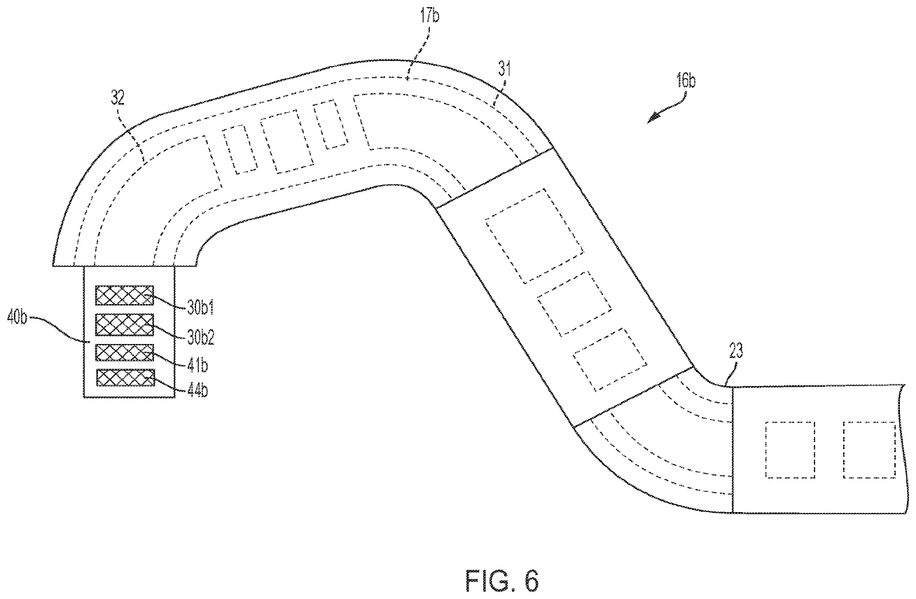

In some implementations there may be more than one scanning magnet. Implementations that include multiple scanning magnets that are at different points along the path of the particle beam and that are separated by air or structures such as magnets or beam-absorbing plates may be referred to as split scanning systems. For example, in beamline structure 16b shown in FIG. 6, which is a variant of beamline structure 30, there may be multiple—for example, two—scanning magnets 30b1 and 30b2 between the between output channel 17b and the treatment position. The scanning magnets may be located in nozzle 40b, along with energy degrader 41b and collimator 44b, on a path of the particle beam between output channel 17b and the treatment position. The scanning magnets may be at separate locations and separated by air or an energy-degrading structure. For example, in this implementation, a first scanning magnet 30b1 may move the particle beam in two dimensions (for example, Cartesian X and Y dimensions) and a second scanning magnet 30b2 may move the particle beam in two dimensions (for example, Cartesian X and Y dimensions). In this example, scanning magnets 30b1 and 30b2 may have the same construction and operation as the scanning magnet shown in FIG. 5. Each magnet 30b1 and 30b2 may move the beam partly, with the combined movements produced by the two magnets producing the desired movement specified in a treatment plan.

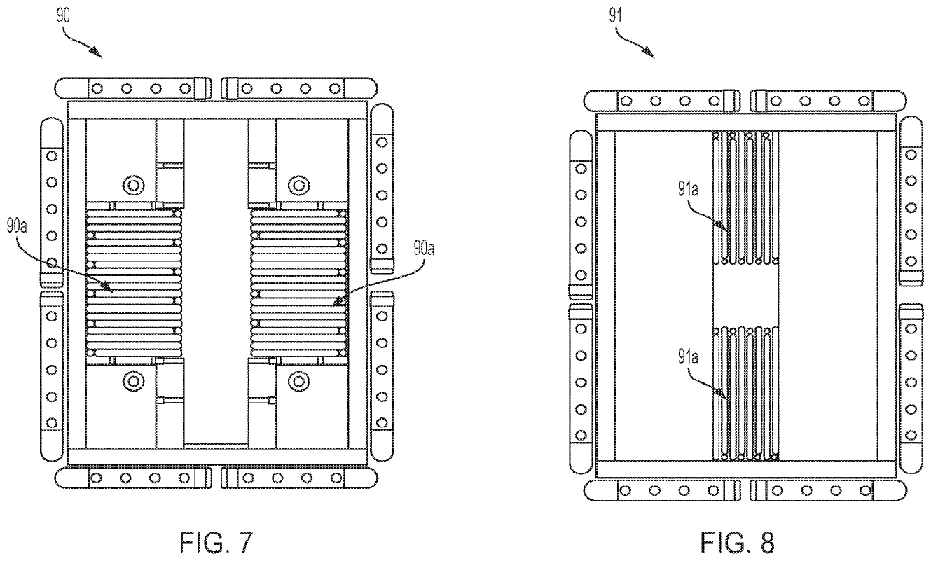

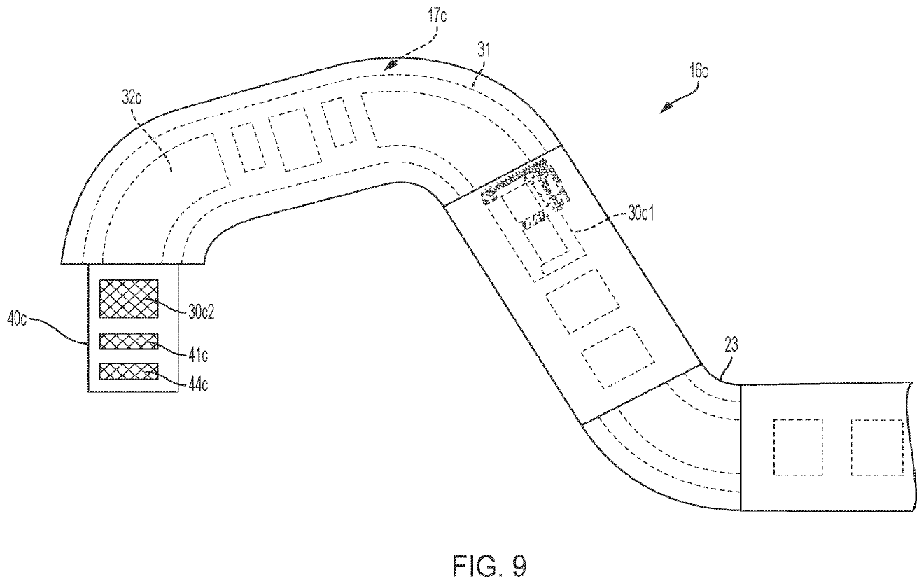

In a variant shown in the FIG. 6 implementation, scanning magnet 30b1 may move the particle beam in one dimension only (for example, the Cartesian X dimension) and scanning magnet 30b2 may move the particle beam in one dimension only (for example, the Cartesian Y dimension). One magnet 30b1 may be upstream of the other magnet 30b2 relative to the particle accelerator as shown in the figure. The two may be separated by air or an energy degrading structure as noted above. FIGS. 7 and 8 show example magnets 90 and 91, respectively, having orthogonal coils—coils 90a are orthogonal to coils 91a—to move the particle beam in different dimensions. In this example, scanning magnet 30b1 may be of the type shown in FIG. 7 and include a first set of coils 90a and scanning magnet 30b2 may be of the type shown in FIG. 8 and include a second set of coils 91a that are orthogonal to coils 90a. Each magnet 30b1, 30b2 may move the beam partly, with the combined movements produced by the two magnets producing the desired movement specified in a treatment plan.

In some implementations, one or more—for example, all or fewer than all—of the scanning magnets may be located in the beamline structure. For example, in beamline structure 16c shown of FIG. 9, which is a variant of beamline structure 16 of FIG. 3, there may be multiple—for example, two—scanning magnets including a first scanning magnet 30c1 located within beamline structure 16c and a second scanning magnet 30c2 located outside of the beamline structure in nozzle 40c, along with energy degrader 41c and collimator 44c between output channel 17 and the treatment position. The first scanning magnet 30c1 may be located among the magnetics included in beamline structure 16c. For example, first scanning magnet 30c1 may be located within output channel 17c upstream of bending magnet 32c relative to the particle accelerator, or as shown in FIG. 9 first scanning magnet 30c1 may be located upstream of output channel 17c relative to the particle accelerator. Bending magnets 32c may be or include a bending magnet of the type shown in, and described with respect to, FIGS. 25 to 31 or a variant thereof. In an example, first scanning magnet 30c1 may be configured to move the particle beam in two dimensions (for example, Cartesian X and Y dimensions) and second scanning magnet 30c2 may be configured to move the particle beam in two dimensions (for example, Cartesian X and Y dimensions). In this example, scanning magnets 30c1 and 30c2 may have the same construction and operation as the scanning magnet shown in FIG. 5. Each magnet 30c1 and 30c2 may move the beam partly, with the combined movements produced by the two magnets producing the desired movement specified in a treatment plan.

In a variant of the FIG. 9 implementation, first scanning magnet 30c1 may be configured to move the particle beam in one dimension only (for example, the Cartesian X dimension) and second scanning magnet 30c2 may be configured to move the particle beam in one dimension only (for example, the Cartesian Y dimension). In this example, scanning magnet 30c1 may include a first set of coils and scanning magnet 30c2 may include a second set of coils that are orthogonal to the first set of coils. Magnets 30c1 and 30c2 may have configurations like the magnets shown in FIGS. 7 and 8 in this example. Each magnet 30c1 and 30c2 may be configured to move the beam partly, with the combined movements produced by the two magnets producing the desired movement specified in a treatment plan.

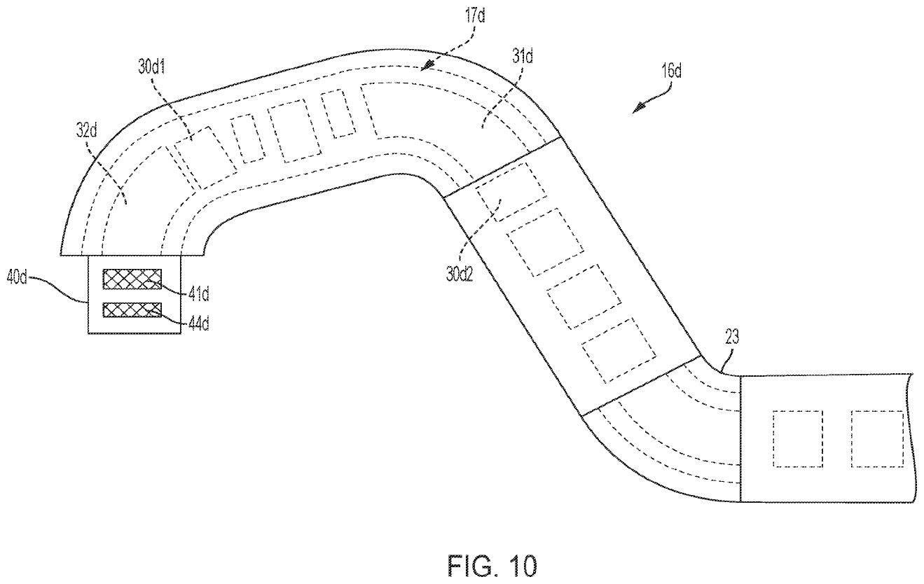

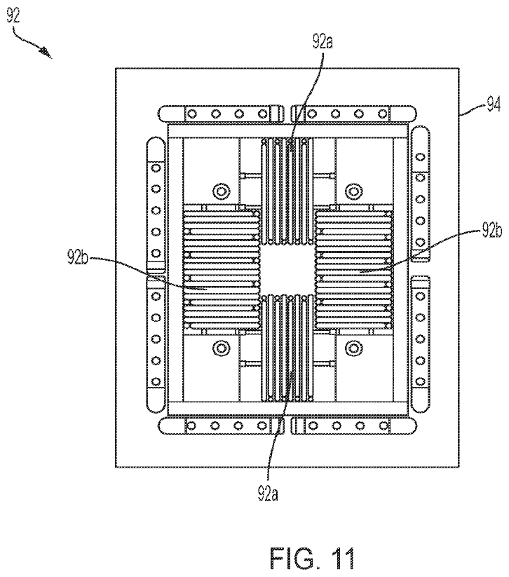

In some implementations, all of the scanning magnets may be located in the beamline structure. As shown in the split scanning system of FIG. 10, both a first scanning magnet 30d1 and a second scanning magnet 30d2 may be located within beamline structure 16d. No scanning magnets may be located in nozzle 40d, which includes energy degrader 41d and collimator 44d in this example. In other examples, there may be one or more scanning magnets also in the nozzle. First scanning magnet 30d1 and second scanning magnet 30d2 may be located among the magnetics included in beamline structure 16d. For example, as shown in FIG. 10 first scanning magnet 30d1 may be located within output channel 17d upstream of magnetic dipole 32d relative to the particle accelerator, or the first scanning magnet may be located upstream of output channel 17d relative to the particle accelerator. Second scanning magnet 30d2 may be located upstream of first scanning magnet 30d1 relative to the particle accelerator. In the example shown in FIG. 10, second scanning magnet 30d2 precedes output channel 17d in the beamline. The scanning magnets may be at separate locations within the beamline structure and separated by magnetics, such as a dipole or quadrupole magnet, and/or air within the beamline structure. The separate locations may include different points or locations in series along a path of the particle beam or length of the beamline structure. For example, as shown in FIG. 10, bending magnet 31d is between first scanning magnet 30d1 and second scanning magnet 30d1. In another example, scanning magnet 30d1 may be moved after bending magnet 32d such that both bending magnets 31d and 32d are between scanning magnets 30d1 and 30d1. In another example, both scanning magnets 30d1 and 30d2 may be within output channel 17d and bending magnets 31d and 32d may be between scanning magnets 30d1 and 30d2. In an example, first scanning magnet 30d1 may be configured to move the particle beam in two dimensions (for example, Cartesian X and Y dimensions) and second scanning magnet 30d2 may be configured to move the particle beam in two dimensions (for example, Cartesian X and Y dimensions). In this example, scanning magnets 30d1 and 30d2 may have the same construction and operation as the scanning magnet shown in FIG. 5. Each magnet 30d1 and 30d2 may move the particle beam partly, with the combined movements produced by the two scanning magnets producing the desired particle beam movement specified in a treatment plan. Bending magnets 23, 31d and 32d may be or include a bending magnet of the type shown in, and described with respect to, FIGS. 25 to 31 or a variant thereof.

In a variant of the FIG. 10 implementation, first scanning magnet 30d1 may be configured to move the particle beam in one dimension only (for example, the Cartesian X dimension) and second scanning magnet 30d2 may be configured to move the particle beam in one dimension only (for example, the Cartesian Y dimension). In this example, scanning magnet 30d1 may include a first set of coils and scanning magnet 30d2 may include a second set of coils that are orthogonal to the first set of coils. Magnets 30d1 and 30d2 may have configurations like the magnets shown in FIGS. 7 and 8 in this example. Each magnet 30d1 and 30d2 may be configured to move the beam partly, with the combined movements produced by the two magnets producing the desired movement specified in a treatment plan.

In some implementations, there may be more than two scanning magnets located within the beamline structure and/or located between the output of the output channel and the treatment position. For example, there may be three or more scanning magnets located at various separate locations within the beamline structure. For example, there may be three or more scanning magnets located at various separate locations between the output of the output channel and the treatment position. In each case, the scanning magnets may be arranged in series.

In some implementations, there may be a single scanning magnet located within the beamline structure upstream of the output of output channel or elsewhere. For example, as shown in FIGS. 2 and 3, scanning magnet 30 may be located upstream of output channel 17 relative to the particle accelerator and at the input of output channel 17. Scanning magnet 30 may be configured to move the particle beam in two dimensions (for example, the Cartesian X and Y dimension). In this example, scanning magnet 30 may have the same construction and operation as the scanning magnet shown in FIG. 5. In this example, all particle beam movement is implemented by controlling current through one or more coils of the single scanning magnet.

In this regard, by positioning all or some of the scanning magnets within a beamline structure, it may be possible to reduce the size of the particle therapy system relative to systems that implement scanning external to the gantry.

In some implementations, one or more the scanning magnets described herein may be superconducting. For example, one or more, including all, of the scanning magnets downstream of the output channel may be superconducting. For example, one or more, including all, of the scanning magnets within the beamline structure may be superconducting. In this regard, it can be difficult to move the particle beam accurately in the presence of high magnetic fields such as those found in the beamline structure. Use of a superconducting magnet for scanning enables generation of magnetic fields of 2.5 T or greater or 3 T or greater to move the particle beam, which can overcome effects on the particle beam of the high magnetic fields, such as 2.5 T or greater or 3 T or greater, produced by the beamline structure.

FIG. 11 shows an example implementation of a superconducting scanning magnet 92 configured to move the particle beam in two dimensions, which may be used in the scanning implementations described herein. In this example, scanning magnet 92 may have the same construction and operation as scanning magnet 43 shown of FIG. 5. Superconducting magnet 92 includes sets of high-temperature superconducting coils 92a and 92b, which are similar in construction to coils 46 and 45, respectively, of FIG. 5. Examples of high-temperature superconductors include, but are not limited to, YBCO (yttrium barium copper oxide) and BSCCO (bismuth strontium calcium copper oxide). Scanning magnet 92 is contained in a cryostat 94 that maintains the superconducting magnet at superconducting temperatures, e.g., above 77° Kelvin (K) or above 90° K. A cryostat may include a device configured to maintain the superconducting coils at cryogenic temperatures. The cryostat may maintain temperature by thermally isolating the superconducting coils from room temperature. This generally is performed using vacuum insulation, thermal radiation shields and/or superinsulation to reduce radiation heat transfer, and low thermal conductivity connections between room temperature and cryogenic temperatures. In some examples, liquid helium may be used to cool the coils to superconducting temperatures in the cryostat using, for example, conductive or immersive cooling. In conductive cooling, heat is transferred away from the superconducting coils using a thermal conductor. In immersive cooling, the superconducting coils may be in direct contact with a cryogen, such as liquid helium. In operation, current is applied to coils 92a and 92b to generate the magnetic fields used for scanning.

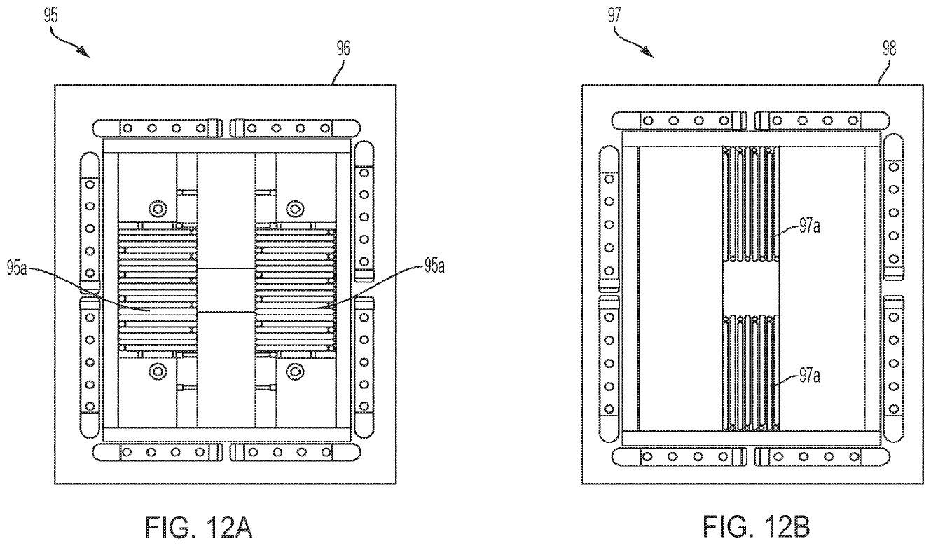

FIG. 12a shows an example of a superconducting magnet 95 configured to move the particle beam in one dimension only, which may be used in scanning implementations described herein. The superconducting magnet includes high-temperature superconducting coil set 95a, which is configured to move the particle beam one dimension only (for example, the Cartesian X or Y dimension). Examples of high-temperature superconductors include, but are not limited to, YBCO and BSCCO. Superconducting magnet 95 is contained in a cryostat 96 that maintains the superconducting magnet at superconducting temperatures, e.g., above 77° Kelvin (K). For example, liquid helium may be used to cool the coils to superconducting temperatures. Current is applied to coils 95a to generate the magnetic fields used for scanning. FIG. 12b shows an example of a superconducting scanning magnet 97 configured to move the particle beam in one dimension only. That dimension is different from, such as orthogonal to, the dimension that magnet 95 of FIG. 12a moves the particle beam. Superconducting magnet 97 includes high-temperature superconducting coil set 97a, which is configured to move the particle beam one dimension only (for example, the Cartesian X or Y dimension). Examples of high-temperature superconductors include, but are not limited to, YBCO and BSCCO. Superconducting magnet 95 is contained in a cryostat 98 that maintains the superconducting magnet at superconducting temperatures, e.g., above 77° Kelvin (K). For example, liquid helium may be used to cool the coils to superconducting temperatures. Current is applied to coils 97a to generate the magnetic fields used for scanning.

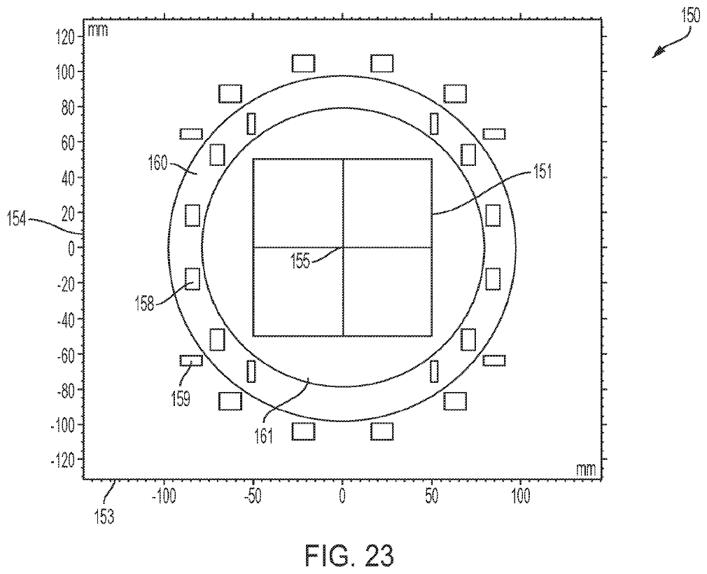

FIG. 23 shows a front, cut-away view of another example implementation of a superconducting scanning magnet 150 configured to move the particle beam in two dimensions, which may be used in the scanning implementations described herein. In this example, scanning magnet 150 may be contained in a cryostat (not shown) such as that described above to maintain the superconducting magnet at superconducting temperatures, e.g., between 30° K and 40° K in this example, although the cryostat is not limited to these temperatures. A cryocooler may be used to maintain the temperature of the cryostat at superconducting temperatures. A cryocooler includes a device for providing active cooling of the superconducting coils down to cryogenic temperatures. The cryocooler may be controlled by the control systems described herein.

In FIG. 23, grid 151 shows the scanning beam aperture in both the Cartesian X and Y dimensions, 153 and 154, respectively. For example, grid 151 shows that scanning magnet 150 can move the particle beam ±5 cm in the X dimension and ±5 cm in the Y dimension relative to a reference 0,0 point 155. In other implementations, the scanning magnet may be configured to move the particle beam over lengths that are more or less than ±5 cm in the X dimension and ±5 cm in the Y dimension. In FIG. 23, sets of superconducting coils 158 and 159 are wound around an electrically nonconductive or an electrically non-superconducting material 160 to create aperture 161 that contains grid 151. Inner superconducting coils 158 may be separated from outer superconducting coils 159 by an electrically nonconductive or an electrically non-superconducting material 160. Superconducting coils 158 may be configured so that the magnetic fields generated thereby are orthogonal to the magnetic fields generated by superconducting coils 159. And, superconducting coils 159 may be configured so that the magnetic fields generated thereby are orthogonal to the magnetic fields generated by superconducting coils 158. For example, the windings of superconducting coils 158 and 159 may be orthogonal to each other. In some implementations, the magnetic fields generated by superconducting coils 158 and 159 need not be orthogonal, but rather may be different—for example, at an angle to each other that is less than 90°—yet still enable scanning in a grid such as grid 151.

In this example, superconducting coils 158 control movement of the particle beam in the X dimension. For example, current runs through those superconducting coils to produce a magnetic field. The strength of that magnetic field is proportional to the amount of current running through the superconducting coils. And, the strength of the magnetic field is proportional to the amount that the particle beam moves in the X dimension during scanning. In this example, superconducting coils 159 control movement of the particle beam in the Y dimension. For example, current runs through those superconducting coils to produce a magnetic field. The strength of that magnetic field is proportional to the amount of current running through the superconducting coils. And, the strength of the magnetic field is proportional to the amount that the particle beam moves in the X dimension during scanning. Current may run through superconducting coils 158 and 159 at the same time to produce a cumulative magnetic field that moves the particle beam in both the X and Y dimensions. Current may run through superconducting coils 158 and 159 at different times so that the particle beam moves in the X or Y dimensions at separate times, but still reaches a target location.

An example of electrically non-superconducting material that may be included in scanning magnet 150 is copper; however, scanning magnet 150 is not limited to use with copper. The electrically non-superconducting material promote heat dissipation, for example during a quench of the superconducting coils 158 and 159.

FIG. 24 shows a cross-section of an example superconducting coil 165 that may be used to implement each of superconducting coils 158 and 159 and/or the coils described with respect to the example bending magnets of FIGS. 25 to 31. Superconducting coil 165 includes a copper (Cu) stabilization layer 166 that encases or surrounds the other layers of superconducting coil 165. Superconducting coil 165 also includes a silver (Ag) cap layer 167, a rare-earth barium copper oxide (ReBCO) superconducting layer 168 (or layer(s) of other high-temperature superconducting material(s)) adjacent to and in contact with the silver cap layer, a buffer layer stack 169 adjacent to and in contact with the ReBCO superconducting layer to prevent interdiffusion between oxides and a metal substrate, and a substrate layer 170 adjacent to and in contact with the buffer layer stack. Examples of materials that may be included in the substrate layer include, but are not limited to, an electrically-conductive metal such as copper, nickel, or aluminum. Examples of materials that may be included in the buffer layer stack include, but are not limited to, SrRuO₃ (strontium ruthenate—SRO) and LaNiO₃ (LNO). Superconducting coil 165 may have a different configuration than that shown or may include different materials than those shown. For example, the copper stabilization layer may be omitted or a material other than copper may be used. Other types of superconducting materials may be used, such as YBCO and/or BSCCO.

Referring back to FIG. 3, output channel 17 portion of beamline structure 16 includes large-aperture superconducting (or non-superconducting) bending magnet 31 arranged in series with large-aperture superconducting (or non-superconducting) bending magnet 32. Examples of large apertures include, but are not limited to 20 cm by 20 cm. Bending magnets 31 and 32 may be or include a bending magnet of the type shown in, and described with respect to, FIGS. 25 to 31 or a variant thereof.

Located between bending magnet 31 and bending magnet 32 are multiple large-aperture superconducting (or non-superconducting) magnetic quadrupoles 33, 34, and 35. In this example, magnetic quadrupoles 33, 34, and 35 include, alternately, one or more focusing magnets and one or more defocusing magnets to focus and defocus the particle beam, respectively, in order to maintain a substantially consistent cross-sectional area of the particle beam. In this regard, the net effect on the particle passing through the alternating magnetic field gradients of the magnetic quadrupoles is to cause the beam to converge; that is, to focus. In some implementations, magnetic quadrupole 33 includes a defocusing magnet, magnetic quadrupole 34 includes a focusing magnet, and magnetic quadrupole 35 includes a defocusing magnet. In some implementations, magnetic 33 includes a focusing magnet, magnetic quadrupole 34 includes a defocusing magnet, and magnetic quadrupole 35 includes a focusing magnet. In some implementations, output channel 17 may include different numbers of magnetic quadrupoles in different configurations and/or a different number of magnetic dipoles in a different configuration. In some implementations, output channel 17 may include higher-order magnetics, such as sextupoles, in place of, or in addition to, the magnetic quadrupoles that are shown.

In some implementations, using bending magnets 31 and 32, output channel 17 is configured to bend the particle beam in the presence of magnetic fields of 2.5 T, 3 T, or greater in the beamline structure. For example, the magnetic fields may be generated by running current through one or more coils in the magnets in the beamline structure, which may be on the order of 2.5 T or more, 3 T or more, 4 T or more, 5 T or more, 6 T or more, 7 T or more, 8 T or more, 9 T or more, 10 T or more, 11 T or more, 12 T or more, 13 T or more, 14 T or more, or 15 T or more. In the presence of magnetic fields such as these, the magnetics in output channel 17 are configured to produce a combined total bending angle of the particle beam anywhere in a range from 90° to 170°—for example, 90°, 95°, 100°, 105°, 110°, 115°, 120°, 125°, 130°, 135°, 140°, 145°, 150°, 155°, 160°, 165°, or 170°. Alternatively, in some implementations, using bending magnets 31 and 32, output channel 17 is configured to bend the particle beam at a combined total bending angle that is less than 90° or that is greater than 170°—for example, 180° or greater. In FIGS. 1 to 3, using bending magnets 31 and 32, output channel 17 is configured to bend the particle beam at a combined total bending angle of about 150° relative to line 38. To achieve a bending magnitude having a value from 110° to 170°, bending magnet 31 may be configured to bend the particle beam within a range of 20° to 85° relative to line 38, and bending magnet 32 may be configured to bend the particle beam within a range of 20° to 85° relative to horizontal line 38.

In some implementations, output channel 17 may include different numbers of magnetic structures in different configurations. For example, output channel 17 may include a bending magnet of the type described herein, followed by three alternating magnetic quadrupoles, followed by a bending magnet of the type described herein, followed by three alternating magnetic quadrupoles, followed by a bending magnet of the type described herein. Additional magnetics may be used, for example, to change where and by how much the particle beam bends. Additional magnetic structures may also be used to focus the particle beam over longer distances. Conversely, fewer numbers of magnetic structures may be used to focus the particle beam over shorter distances, as shown in FIG. 1 for example.

A nozzle 40 (FIG. 1) is located at the output or exit of output channel 17. In the example of FIG. 1, nozzle 40 is connected to output channel 17 and, where applicable, moves along with output channel. Nozzle 40 may, or may not, be considered to be part of the compact gantry. Nozzle 40 is an example of a particle beam output device. In this example, nozzle 40 receives the particle beam from output channel 17 and, in some implementations, conditions the particle beam for output to an irradiation target, such as a tumor in a patient, at the treatment position or isocenter. In this regard, as noted, output channel 17 bends the particle beam by at least 90°. The particle beam is thus directed towards the treatment position or isocenter as it exits output channel 17. In addition, as described herein, scanning magnet(s) 30 may move the particle beam within a plane to move the particle beam across the irradiation target.

In this regard, as explained previously, the nozzle may contain one or more scanning magnets. The energy degrader is downstream of the scanning magnets and the collimator is downstream of the scanning magnets. In FIGS. 2 and 3, energy degrader 41 receives the scanning or moving particle beam from the scanning magnet(s). In this example, energy degrader 41 is mounted to gantry 14 (via nozzle 40) between output channel 17 and the irradiation target at treatment position 19. Energy degrader 41 is configured to, and controllable to, change an energy of the particle beam before the particle beam reaches the irradiation target. In some implementations, the energy degrader is the sole mechanism by which to actively control the change in energy of the particle beam prior to the particle beam reaching the irradiation target. In some implementations, the energy of the particle beam is not actively controllable after the particle beam is output by the particle accelerator and prior to the particle beam reaching the energy degrader. For example, in such implementations, components of the gantry between the particle accelerator and the energy degrader do not, and are not configured to, actively control the beam energy. Stated yet another way, the gantry or the beamline conduit thereof is not configured to actively control the particle beam after the particle beam is output by the particle accelerator and prior to the particle beam reaching the energy degrader. In some cases, there may be some incidental changes in energy caused by movement through the beamline structure; however, those changes are not actively controlled.

Referring to FIG. 3, in some implementations, a single quadrupole magnet may be used in place of quadrupoles 21, 22; a quadrupole magnet may replace scanning magnet 30; scanning magnet 30 may replace quadrupole magnet 35; and the nozzle may include a second scanning magnet. The scanning magnets in this case may each scan in two dimensions or in one dimension as described herein, or one may scan in two dimensions and one may scan in one dimension.

As noted previously, the particle beam output by the accelerator may be monoenergetic and the energy degrader is the only/sole or primary vehicle for changing beam energy during treatment of an irradiation target. A monoenergetic particle beam includes a particle beam having a single, fixed energy level, such as 100 MeV, 150 Mev, 200 Mev, 250 Mev, and so forth. A monoenergetic particle beam may deviate from the fixed energy level by a predetermined amount, such as ±10%, ±5%, ±2%, or ±1%, and still be considered monoenergetic. Switching operation of the accelerator during treatment, as is required to switch particle beam energies during treatment, may produce excess stray neutrons, resulting in the need for increased shielding and reducing beamline efficiency. The neutrons may be generated by the particle accelerator and/or by magnetics along the beamline structure. By using a particle beam that is monoenergetic during treatment and relying on the energy degrader to change beam energy, production of stray neutrons may be reduced or minimized and the efficiency of the beamline structure may be increased.

In an example, the energy degrader may include plates that are movable into or out of a path of the particle beam. In another example, the energy degrader may include wedges that overlap at least in part and that are movable within a path of the particle beam. An example wedge is a polyhedron defined by two triangles and three trapezoidal faces. In either configuration, variable amounts of material are movable into the path of the particle beam. The material absorbs energy from the particle beam, resulting reduced-energy beam output. The more material there is in the path of the particle beam, the less energy that the particle beam will have. In some implementations, the energy-absorbing structures are movable across all of the beam field or across only part of the beam field. As noted, in some examples, the beam field includes the maximum extent that the particle beam can be moved across a plane parallel to the treatment area on a patient for a given position of the compact gantry.

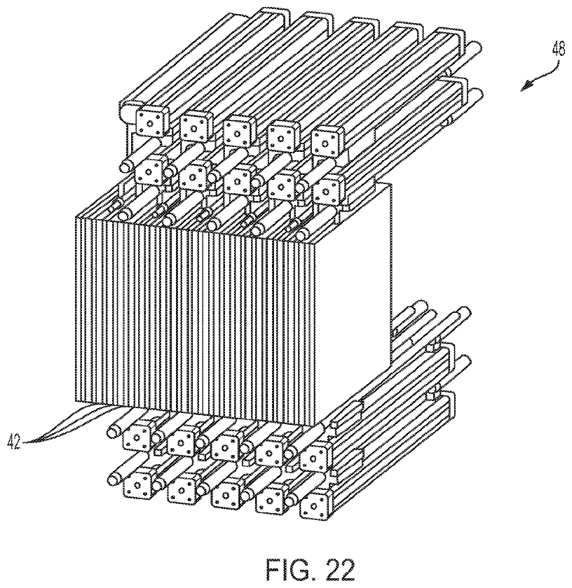

Referring to FIG. 22, in an example, energy degrader 48 is a range modulator that is controllable to move structures 42 into, and out of, the path of the particle beam to change the energy of the particle beam and therefore the depth to which dose of the particle beam will be deposited in the irradiation target. Examples of such energy-absorbing structures include, but are not limited to, plates; polyhedra such as wedges, tetrahedra, or toroidal polyhedra; and curved three-dimensional shapes, such as cylinders, spheres, or cones. In this way, the energy degrader can cause the particle beam to deposit doses of radiation in the interior of an irradiation target to treat layers or columns of the target. In this regard, when protons at a particular energy move through tissue, the protons ionize atoms of the tissue and deposit a dose primarily at a predefined tissue depth corresponding to that energy. The energy degrader thus is configured to move the particle beam in the Cartesian Z dimension through the target, thereby enabling the scanning magnet to perform scanning in a third dimension (Cartesian Z) in addition the Cartesian X and Y dimensions. In some implementations, an energy absorbing structure of the energy degrader, such as a plate or wedge, may be configured to move during movement (scanning) of the particle beam and track or trail the particle beam during movement. An example energy degrader that tracks or trails particle beam movement is described in U.S. Pat. No. 10,675,487 (Zwart) entitled “High-Speed Energy Switching”. The content of U.S. Pat. No. 10,675,487, particularly the content related to the energy degrader that tracks or trails particle beam movement (e.g., FIGS. 36 to 46 of U.S. Pat. No. 10,675,487 and the accompanying description), is incorporated herein by reference.

The Bragg peak is a pronounced peak on the Bragg curve that plots the energy loss of ionizing radiation during travel through tissue. The Bragg peak represents the depth at which most radiation deposits within tissue. For protons, the Bragg peak occurs right before the particles come to rest. Accordingly, the energy of the particle beam may be changed to change the location of its Bragg peak and, therefore, where a majority of the dose of protons will deposit in depth in the tissue. In this regard, the particle accelerator may be a fixed-energy particle accelerator. In a fixed-energy particle accelerator, the particle beam always exits the particle accelerator at the same, or about the same, energy—for example, within a 10%, 5%, or 1% deviation or less from an expected or target energy. In a fixed-energy particle accelerator, the energy degrader is the primary vehicle or the sole vehicle for varying the energy of the beam applied to an irradiation target in the patient. In some implementations, the particle accelerators described herein are configured to output particle beams at a single energy or at two or more energies within a range between about 100 MeV and about 300 MeV (for example, between 115 MeV and 250 MeV). The fixed energy output may be within that range (e.g., 250 MeV) or, in some examples, above or below that range.

In some implementations, the particle accelerator is a dual-energy accelerator. In a dual-energy particle accelerator, the particle beam exits the particle accelerator at one of two different energy levels-a high energy level or a low energy level. The terms “high” and “low” have no specific numerical connotations but rather are intended to convey relative magnitudes. In some implementations, the particle accelerators described herein are configured to output particle beams at two energies that are within a range that is between about 100 MeV and about 300 MeV. The high energy output and the low energy output may be values within that range or, in some examples, above or below that range. The energy degrader described herein may be used with dual-energy particle accelerators in order to reduce the energy of the particle beam below one of the two energy levels and/or to finely adjust between the two energy levels.

In the figures, nozzle 40 also includes a collimator 44 downstream of energy degrader 41 relative to the particle accelerator (that is, closer to the irradiation target). In an example, a collimator is a structure that is controllable to allow some radiation to pass to a target and to block some radiation from passing to the patient. Typically, the radiation that passes is directed to an irradiation target to be treated, and the radiation that is blocked would otherwise hit, and potentially damage, healthy patient tissue. In operation, the collimator is placed in the radiation path between output channel 17 and the irradiation target and is controlled to produce an opening of an appropriate size and shape to allow some radiation to pass through the opening to the irradiation target, while a remainder of the structure blocks some radiation from reaching adjacent tissue.

The collimator may be configurable—for example, its aperture may be controlled and changed during treatment. The collimator may be fixed or not changeable. For example, the collimator may have a fixed shape that cannot be altered.

In some implementations, components of an example configurable collimator include multiple leaves that are dynamically reconfigurable during movement of the particle beam to change a shape of an edge defined by the multiple leaves. The edge is movable between at least a portion of the particle beam and a target of the particle beam so that a first part of the particle beam on a first side of the edge is at least partly blocked by the multiple leaves and so that a second part of the particle beam on a second side of the edge is allowed to pass to the target.

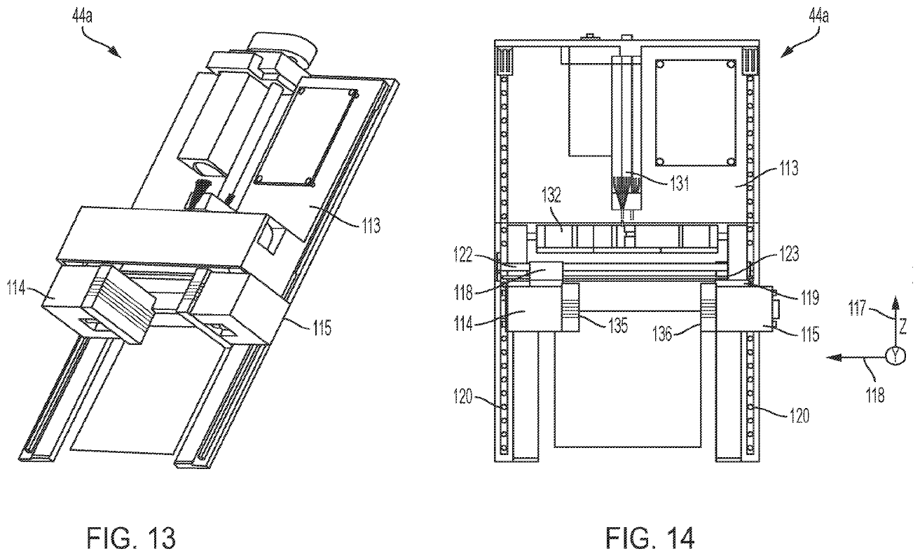

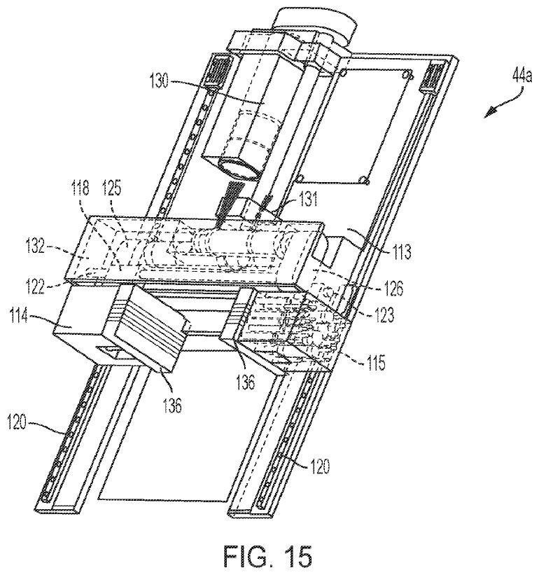

FIGS. 13, 14, and 15 show an example implementation of configurable collimator 44a, which may be used with the particle therapy system described herein. Collimator 44a including carriages 113, 114, and 115 configured to hold, and to move, the leaves described above both vertically and horizontally relative to an irradiation target. As shown, vertical movement includes movement in the Cartesian Z-dimension 117, and horizontal movement includes movement in the Cartesian X dimension 118 (with the Cartesian Y dimension being into, or out of, the page in FIGS. 13 and 14). FIGS. 14 and 15 show parts of carriage housings as transparent in order to show components inside the housings; however, the housings are not actually transparent.

Carriage 113 is referred to herein as the primary carriage, and carriages 114 and 115 are referred to herein as secondary carriages. Secondary carriages 114, 115 are coupled to primary carriage 113, as shown in FIGS. 13 to 15. In this example, secondary carriages 114, 115 each include a housing that is fixed to primary carriage 115 via a corresponding member 118, 119. In this example, primary carriage 113 is movable vertically (the Z dimension) relative to the irradiation target and relative to particle accelerator along tracks 120. The vertical movement of primary carriage 113 also causes the secondary carriages to move vertically. In some implementations, the secondary carriages move vertically in concert.

As shown in FIGS. 13 to 15, each secondary carriage 114, 115 is connected to a corresponding rod or rail 122, 123, along which the secondary carriage moves. More specifically, in this example, motor 125 drives secondary carriage 114 to move along rod 122 towards or away from secondary carriage 115. Likewise, in this example, motor 126 drives secondary carriage 115 to move along rod 123 towards or away from secondary carriage 114. Control over movement of the primary and secondary carriages is implemented to position the leaves relative to the irradiation target, as described herein. In addition, the leaves themselves are also configured to move in and out of the carriages, as also described herein.

As shown in FIG. 15, a motor 130 drives the vertical movement of primary carriage 113. For example, as shown in FIG. 15, lead screw 131 is coupled to housing 132, which holds motors 125, 126 that drive corresponding secondary carriages 114, 115, and which is mounted on tracks 120. Lead screw 131 is coupled to, and driven vertically by, motor 130. That is, motor 130 drives lead screw 131 vertically (the Cartesian Z dimension). Because lead screw 131 is fixed to housing 132, this movement also causes housing 132, and thus secondary carriages 114, 115, to move along tracks 120, either towards or away from the irradiation target.

In this example implementation, seven leaves 135, 136 are mounted on each secondary carriage 114, 115. Each secondary carriage may be configured to move its leaves horizontally into, or out of, the treatment area. Using linear motors, the individual leaves on each secondary carriage may be independently and linearly movable in the X dimension relative to other leaves on the same secondary carriage. In some implementations, the leaves may also be configured to move in the Y dimension. Furthermore, the leaves on one secondary carriage 114 may be movable independently of the leaves on the other secondary carriage 115. These independent movements of leaves on the secondary carriages, together with the vertical movements enabled by the primary carriage, allow the leaves to be moved into various configurations. As a result, the leaves can conform, both horizontally and vertically, to treatment areas that are randomly shaped both in horizontal and vertical dimensions. The sizes and shapes of the leaves may be varied to create different conformations. For example, the sizes and shapes may be varied to treat a single beam spot and, thus, a single column. In some implementations individual leaves on each secondary carriage may be independently and linearly movable using electric motors that drive lead screws in the X dimension relative to other leaves on the same secondary carriage.

The leaves may be made of any appropriate material that prevents or inhibits transmission of radiation. The type of radiation used may dictate what material(s) are used in the leaves. For example, if the radiation is X-ray, the leaves may be made of lead. In the examples described herein, the radiation is a proton or ion beam. Accordingly, different types of metals or other materials may be used for the leaves. For example, the leaves may be made of nickel, tungsten, lead, brass, steel, iron, or any appropriate combinations thereof. The height of each leaf may determine how well that leaf inhibits transmission of radiation.

Implementations of the configurable collimator described with respect to FIGS. 13 to 15 are described in U.S. Patent Publication No. 2017/0128746 (Zwart) entitled “Adaptive Aperture”. The content of U.S. Patent Publication No. 2017/0128746, particularly the content relating to the description of the adaptive aperture (e.g., FIGS. 1 to 7 of U.S. Patent Publication No. 2017/0128746 and the accompanying description), is incorporated herein by reference.

Referring back to FIG. 1, as noted, example particle therapy system include an isocentric gantry that is compact in size, which reduces overall system size. In implementations of compact gantry 14, the diameter of support structure 15 may be less than 6 meters (m), less than 5 m, or less than 4 m. In an example, the diameter of support structure 15 is 4.8 m. The length of the beamline structure may be measured from, and equal to the distance between, the output of the accelerator and the system isocenter. In implementations of compact gantry 14, the length of beamline structure 16 may be less than 6 meters (m), less than 5 m, less than 4.5 m, or less than 4 m. In an example, the length of beamline structure 16 is 4.2 m (FIG. 2). In this regard, the distance between the particle accelerator and the system isocenter or treatment position may be less than 6 m, less than 5 m, less than 4.5 m, or less than 4 m. In implementations of compact gantry 14, the distance between the output of output channel 17 and the system isocenter or the treatment position is 2 m or less, 1.5 m or less, or 1 m or less. In implementations of compact gantry 14, the distance between the output of output channel 17 and the system isocenter or the treatment position is between 0.8 m and 1.4 m. In an example, the distance between the output of output channel 17 and the system isocenter or the treatment position is 1.01 m (FIG. 2). Other implementations may have different dimensions than those listed here.

In some implementations, the particle therapy system has a footprint of 93 square meters (m²) or less or of 75 m² or less. In some implementations, the particle therapy system is configured to fit within a vault designed for a LINAC. For example, the components of FIGS. 1 to 3 may be small enough fit within, and have dimensions that fit within, a vault having the following dimensions: 25 feet (7.62 m) or less in length, 20 feet (6.09 m) or less in width, and 11 feet (3.35 m) or less in height. For example, the components of FIGS. 1 to 3 may be small enough fit within, and have dimensions that fit within, a vault having the following dimensions: 25 feet (7.62 m) or less in length, 26 feet (7.92 m) or less in width, and 10 feet (3.05 m) or less in height. For example, the components of FIGS. 1 to 3 may be small enough fit within, and have dimensions that fit within, a LINAC vault having a footprint of 26.09 feet (11 m) or less by 29.62 feet (9 m) or less, with a height of 16.40 feet (5 m) or less. However, as noted, some implementations of the particle therapy system may have different dimensions including, but not limited to, diameters, heights, widths, and lengths. In some implementations, the ceiling of a pre-existing LINAC vault may not be high enough to support full 360° rotation of or around the gantry. In such implementations, a pit 90 (FIG. 1) may be dug beneath the floor of the LINAC vault to enable the rotation.

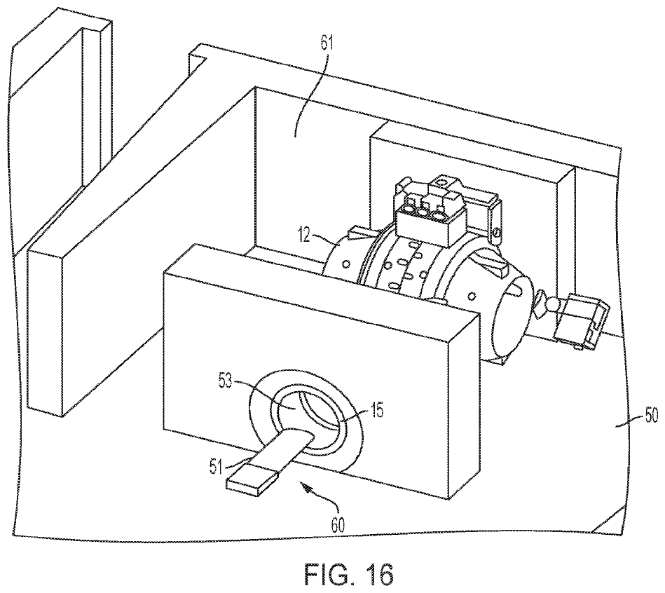

FIGS. 1 and 16 shows examples of treatment spaces 49 and 50 in which particle therapy system 10 and its variants may be housed. The treatment spaces are implemented in LINAC vaults in these examples, which may be shielded using lead or other appropriate materials such as concrete, borated polyethylene, and/or steel. In this regard, particles, such as protons, that are created by the particle accelerator but do not reach the irradiation target create secondary radiation through the production of high energy neutrons. In an example, particle accelerator 12 and/or the gantry generates 10 millisieverts or less of such neutrons per gray of dose delivered by the particle beam.

Use of a monoenergetic particle bean and reliance on an energy degrader that is outside of the beamline structure enables the magnetics in the beamline to direct the beam efficiently. More specifically, changes in beam energy within the beamline increase production of stray neutrons and, therefore, losses of particle beam within the beamline, thereby degrading its efficiency. The monoenergetic particle beam used in the implementations of the systems described herein, combined with the magnetic structures in the beamline, may lead to increased efficiency. In some cases, decreases in the length of the beamline structure may also increase efficiency. In some implementations, the variants of the beamline structure described herein have an efficiency of 10% or more, 20% or more, 30% or more, 40% or more, 50% or more, 60% or more, 70% or more, 80% or more, or 90% or more. In some examples, efficiency is a measure of the percentage of particles output from the particle accelerator that are output from the beamline structure. So, an efficiency of 10% or more includes 10% or more of the particles output from the particle accelerator being output from the beamline structure; an efficiency of 20% or more includes 20% or more of the particles output from the particle accelerator being output from the beamline structure; an efficiency of 30% or more includes 30% or more of the particles output from the particle accelerator being output from the beamline structure; an efficiency of 40% or more includes 40% or more of the particles output from the particle accelerator being output from the beamline structure; an efficiency of 50% or more includes 50% or more of the particles output from the particle accelerator being output from the beamline structure; an efficiency of 60% or more includes 60% or more of the particles output from the particle accelerator being output from the beamline structure; an efficiency of 70% or more includes 70% or more of the particles output from the particle accelerator being output from the beamline structure; an efficiency of 80% or more includes 80% or more of the particles output from the particle accelerator being output from the beamline structure; and an efficiency of 90% or more includes 90% or more of the particles output from the particle accelerator being output from the beamline structure. In an example, the particle accelerator and gantry described herein transmit more than 70% of a proton beam to a patient even at energies in lower range of the accelerator.

Beamline efficiency of the type described herein enables a “single room” solution in which the particle accelerator, the gantry, and patient all reside with a single vault, as described above. Within this vault, the particle accelerator itself may include shielding, but separate compartments 60 and 61 (see FIG. 16) in the vault containing the patient and the particle accelerator, respectively, need not be shielded from each other. In other words, in some implementations, there is no electromagnetic shielding that is external to the particle accelerator and the gantry that separates the particle accelerator from the patient. Shielding may not be needed due to the low levels of neutrons emitted by the system. In some implementations, there may be minimal shielding between the separate compartments 60 and 61. For example, the shielding may be 30 cm or less in thickness, 20 cm or less in thickness, or 10 cm or less in thickness.

Referring also to FIG. 1, particle therapy system 10 also includes a treatment couch 51. Treatment couch 51 is configured to move relative to hole 53 in or through gantry 14 to position a patient at the system isocenter or treatment position. In this example, treatment couch 51 is mounted to a robotic arm 54. Arm 54 includes a first segment 55, a second segment 56, and third segment 57. First segment 55 is rotatably coupled to second segment 56 and second segment 56 is rotatably coupled to third segment 57. Treatment couch 51 is coupled to third segment 57 as shown in the figure. Arm 54 is controllable to move treatment couch 51 in and through hole 53 to position a patient lying on the couch for treatment; that is, to move the patient into the treatment position. In some implementations, arm 54 may position the patient in two degrees of freedom, in three degrees of freedom, in four degrees of freedom, in five degrees of freedom, or in six degrees of freedom. An example of two degrees of freedom is forward-backward movement and left-right movement; an example of three degrees of freedom is forward-backward movement, left-right movement, and up-down movement; an example of four degrees of freedom is forward-backward movement, left-right movement, up-down movement and one of pitch, yaw, or roll movement; an example of five degrees of freedom is forward-backward movement, left-right movement, up-down movement and two of pitch, yaw, or roll movement; and an example of six degrees of freedom is forward-backward movement, left-right movement, up-down movement, pitch movement, yaw movement, and roll movement. In some implementations, the treatment couch may be replaced by or include a couch that inclines at least in part or that is convertible to a chair, and that is still be controllable in two, three, four, five, or six degrees of freedom to position the patient for treatment. In some implementations, arm 54 may have a different configuration than that shown in FIG. 1. For example, arm 54 may have two segments or more than three segments. Hydraulics, robotics, or both, may control or implement non-planar movement of the treatment couch.

In some implementations, output channel 17 may rotate at least part-way, including all the way, around support structure 15 or output channel may remain fixed on support structure 15 and all or part of support structure 15 may rotate around the treatment position. In some implementations, output channel 17 may not rotate around support structure 15 and the support structure may not rotate around the patient. Instead, the output channel may remain stationary, thereby providing a particle beam that is fixed in one direction. In implementations such as these, the treatment couch or other seat moves relative to the fixed beam during treatment. In some system described herein, the location of the particle beam may be set through rotation of the gantry, after which the beam remains fixed except for scanning movements across the irradiation target and the treatment couch or other seat moves during treatment. In some implementations, treatment may be implemented using a combination of gantry movement and treatment couch (or other seat movement). For example, the output channel may be positioned and the beam may be fixed temporarily, during which time the treatment couch moves to implement treatment. After that, the output channel may be repositioned to fix the beam temporarily at a new position. Treatment may be implemented at the new position through couch movement. These operations may be repeated as defined by a treatment plan drafted for use with the particle therapy system.

Particle therapy system 10 may be an intensity-modulated proton therapy (IMPT) system. IMPT systems enable spatial control of circumscribed beams of protons that may have a variable energy and/or intensity. IMPT takes advantage of the charged-particle Bragg peak—as noted, the characteristic peak of dose at the end of particles' delivery range—combined with the modulation of particle beam variables to create target-local modulations in dose that achieve objectives set forth in a treatment plan. IMPT may involve directing particle beams toward the irradiation target at different angles and at different intensities to treat the target. In some implementations, the particle beam may be scanned—for example, moved—across layers of the irradiation target, with each layer being treated one or more times from the same or different angles. Movement across the irradiation target to implement scanning may be performed using the scanning magnet(s) described herein.

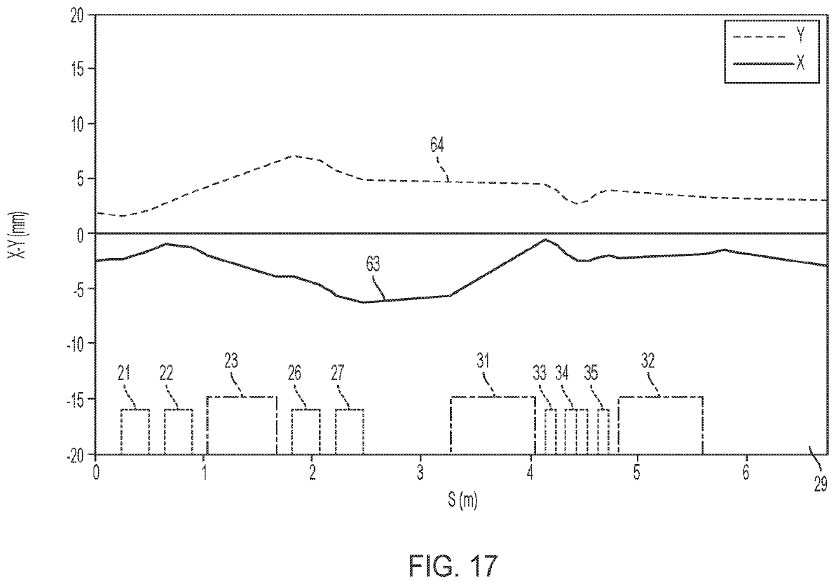

FIG. 17 shows example horizontal (x) beam envelope 63 and vertical (y) beam envelope 64 (e.g., cross-sections) along the length 29 the compact gantry described herein. The x and y dimensions of the beam spot cross-section are determined for magnetic quadrupoles 21 and 22, magnetic dipole 23, magnetic quadrupoles 26 and 27, magnetic dipole 31, magnetic quadrupoles 33, 34, and 35, and magnetic dipole 32. Beam sizes are determined based on calculations of beam optics using measured beam parameters at the exit of particle accelerator 12 and the design parameters of all the beamline magnets. In some implementations, the beam spot radius at the isocenter (e.g., a treatment positions) is approximately 3 millimeters (mm) for both x and y. In some implementations, for 200 MeV to 230 MeV proton beams, magnetic fields at the magnetic dipoles in beamline structure 16 are no more than 4 T and the bending radius of the beam at each of the magnetic dipoles is approximately 0.6 meters. In some implementations, for 200 MeV to 230 MeV proton beams, magnetic fields at the magnetic dipoles in beamline structure 16 are at least 3 T, that is, 3 T or greater. As noted, the systems described herein are not limited to these parameter values and some implementations may have different dimensions, energies, and magnetic fields.

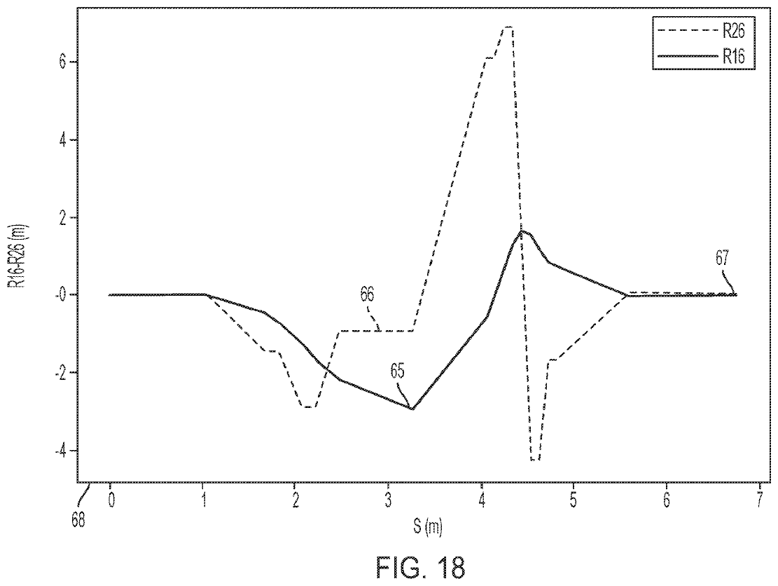

Chromatic-aberration correction can occur in a beamline having dispersion, generated by inclusion of dipole magnets and multiple correctors in dispersive regions. The standard definition for an achromat is a beam transport line having zero values for spatial dispersion (R16) and angular dispersion (R26). Referring to FIG. 18, the magnetics in implementations of the compact gantry may be configured to be achromat—e.g., both R16 65 and R26 66 of the beam transfer matrix elements equal zero at the isocenter, which is at or near location 67, and at the beam entry point to the gantry at 0 m along the beamline structure length 68 (the X-axis). Thus, the gantry as a whole defines an achromat from the beam entry point to the isocenter. Individual magnets or combinations of magnets within the gantry, which constitute less than an entirety of magnets within the gantry along the beamline, need not be achromats. Reducing or minimizing spatial and angular beam dispersions may be consequential to pencil beam scanning techniques implemented by the particle therapy systems described herein. In this regard, in some pencil beam scanning techniques, the cross-section of the particle beam is required to be substantially round at the isocenter. As such, the beam spot size in both x and y (FIG. 18) planes should be close at the isocenter 67. During beam scanning, changes to the beam shape and beam diameter over the entire scanning area should be reduced or minimized, otherwise, different beam particles of different energies may land at different locations in the bending plane. This may cause the beam shape and beam size to differ in another plane.

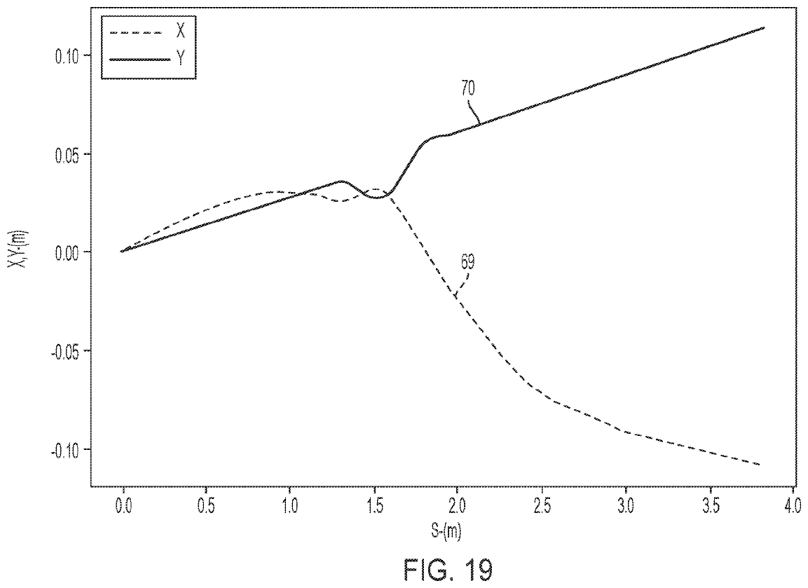

FIG. 19 shows examples of beam scans in the x dimension 69 and the y dimension 70. The firing of the scanning magnets allows the beam particle to be deflected to an angle proportional to the field strength of the scanning magnets. In the example of FIG. 19, a beam scanning range that fully covers a beam field area of 20 cm by 20 cm is shown with beam deflection angles of approximately ±20 milliradians (mrad) and ±30 mrad from the scanning magnets. In this example, the source-to-isocenter distance (SAD) (that is, the accelerator to isocenter distance) is approximately 4 meters. In some implementations, from the scanning magnet(s) to the exit of output channel 17, the beam bending angle can be as large as 110° to 170°.

Referring back to FIG. 1, in some implementations, an imaging system comprised of one or more imaging devices 99 may be mounted to support structure 15. Imaging may be performed before and/or during treatment to identify a target location within the patient and/or to control operation of the gantry and scanning in order to direct the particle beam to the irradiation target in the patient. The imaging system may include one or more of: a computerized tomography (CT) scanner, a two-dimensional (2D) X-ray device, a magnetic resonance imaging (MRI) device, a fan-beam CT scanner, a 2D camera, a three-dimensional (3D) camera, a surface imaging device, or a cone-beam CT scanner

The imaging devices may be configured and controlled to rotate around gantry 14 or to rotate along with rotation of gantry 14. In some implementations, one or more nozzles are rotatable on a ring bearing located at the inner diameter of support structure 15. A variety of two-dimensional (2D) and/or three-dimensional (3D) imaging devices also may be mounted on the ring bearing and may be rotatable therewith. In some implementations, the nozzles and imaging devices may be mounted to different internal circumferential tracks within the gantry. For example, nozzles may be rotatable around a circumferential track at a first radius of the support structure, and imaging devices may be rotatable around a different circumferential track at a second radius of the support structure that is different from the first radius. In some implementations, the gantry may include different rotatable inner rings, one of which mounts the nozzles for rotation and one of which mounts the imaging devices or systems for rotation.

In some implementations, two 2D imaging devices are mounted to support structure 15 in orthogonal planes to enable 2D image-guided radiation therapy (IGRT). IGRT includes the use of imaging during radiation treatment to improve the precision and accuracy of treatment delivery. IGRT may be used to treat tumors in areas of the body that move, such as the lungs. The 2D imaging devices can be rotated to enable cone-beam CT imaging, including simultaneously acquired dual energy imaging. The imaging devices may also, or alternatively, include an X-ray source and an image panel for cone-beam CT image acquisition or a fan-beam diagnostic quality CT imaging device. Alternatively, one plane may include a cone-beam CT imaging device and another plane may include a fan-beam diagnostic quality CT imaging device.

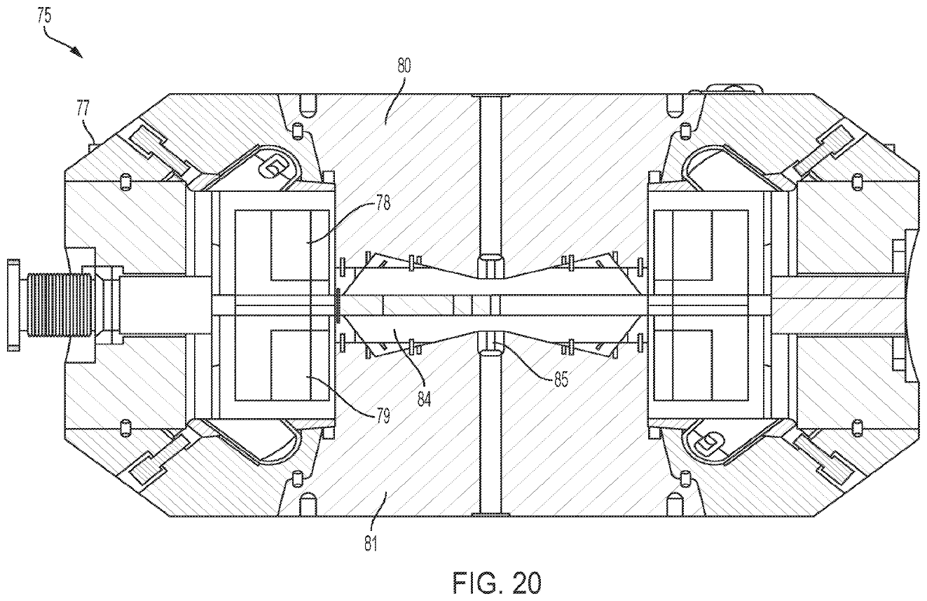

As described herein, an example proton therapy system scans a proton beam in three dimensions across an irradiation target in order to destroy malignant tissue. FIG. 20 shows a cross-section of components 75 of an example superconducting synchrocyclotron that may be used to provide a particle (e.g., a proton) beam in the proton therapy system. In this example, components 75 include a superconducting magnet 77. The superconducting magnet includes superconducting coils 78 and 79. The superconducting coils are formed of multiple integrated conductors, each of which includes superconducting strands-for example, four strands or six strands-wound around a center strand which may itself be superconducting or non-superconducting. Each of the superconducting coils 78, 79 is for conducting a current that generates a magnetic field (B). The magnetic yokes 80, 81 or smaller magnetic pole pieces shape that magnetic field in a cavity 84 in which particles are accelerated. In an example, a cryostat (not shown) uses liquid helium (He) to conductively cool each coil to low-temperature superconducting temperatures, e.g., around 4° Kelvin (K).

In some implementations, the particle accelerator includes a particle source 85, such as a Penning Ion Gauge—PIG source, to provide an ionized plasma column to cavity 84. Hydrogen gas, or a combination of hydrogen gas and a noble gas, is ionized to produce the plasma column. A voltage source provides a varying radio frequency (RF) voltage to cavity 84 to accelerate particles from the plasma column within the cavity. As noted, in an example, the particle accelerator is a synchrocyclotron. Accordingly, the RF voltage is swept across a range of frequencies to account for relativistic effects on the particles, such as increasing particle mass, when accelerating particles within the acceleration cavity. The RF voltage drives a dee plate contained within the cavity and has a frequency that is swept downward during the accelerating cycle to account for the increasing relativistic mass of the protons and the decreasing magnetic field. A dummy dee plate acts as a ground reference for the dee plate. The magnetic field produced by running current through the superconducting coils, together with sweeping RF voltage, causes particles from the plasma column to accelerate orbitally within the cavity and to increase in energy as a number of turns increases. The particles in the outermost orbit are directed to an extraction channel (not shown) and are output from the synchrocyclotron as a particle beam. In a synchrocyclotron, the particle beam is pulsed such that bunches of particles are output periodically.

The magnetic field in the cavity is shaped to cause particles to move orbitally within the cavity as described above. The example synchrocyclotron employs a magnetic field that is uniform in rotation angle and falls off in strength with increasing radius. In some implementations, the maximum magnetic field produced by the superconducting (main) coils may be within the range of 2.5 T to 20 T at a center of the cavity, which falls off with increasing radius. For example, the superconducting coils may be used in generating magnetic fields at, or that exceed, one or more of the following magnitudes: 2.5 T, 3.0 T, 3.1 T, 3.2 T, 3.3 T, 3.4 T, 3.5 T, 3.6 T, 3.7 T, 3.8 T, 3.9 T, 4.0 T, 4.1 T, 4.2 T, 4.3 T, 4.4 T, 4.5 T, 4.6 T, 4.7 T, 4.8 T, 4.9 T, 5.0 T, 5.1 T, 5.2 T, 5.3 T, 5.4 T, 5.5 T, 5.6 T, 5.7 T, 5.8 T, 5.9 T, 6.0 T, 6.1 T, 6.2 T, 6.3 T, 6.4 T, 6.5 T, 6.6 T, 6.7 T, 6.8 T, 6.9 T, 7.0 T, 7.1 T, 7.2 T, 7.3 T, 7.4 T, 7.5 T, 7.6 T, 7.7 T, 7.8 T, 7.9 T, 8.0 T, 8.1 T, 8.2 T, 8.3 T, 8.4 T, 8.5 T, 8.6 T, 8.7 T, 8.8 T, 8.9 T, 9.0 T, 9.1 T, 9.2 T, 9.3 T, 9.4 T, 9.5 T, 9.6 T, 9.7 T, 9.8 T, 9.9 T, 10.0 T, 10.1 T, 10.2 T, 10.3 T, 10.4 T, 10.5 T, 10.6 T, 10.7 T, 10.8 T, 10.9 T, 11.0 T, 11.1 T, 11.2 T, 11.3 T, 11.4 T, 11.5 T, 11.6 T, 11.7 T, 11.8 T, 11.9 T, 12.0 T, 12.1 T, 12.2 T, 12.3 T, 12.4 T, 12.5 T, 12.6 T, 12.7 T, 12.8 T, 12.9 T, 13.0 T, 13.1 T, 13.2 T, 13.3 T, 13.4 T, 13.5 T, 13.6 T, 13.7 T, 13.8 T, 13.9 T, 14.0 T, 14.1 T, 14.2 T, 14.3 T, 14.4 T, 14.5 T, 14.6 T, 14.7 T, 14.8 T, 14.9 T, 15.0 T, 15.1 T, 15.2 T, 15.3 T, 15.4 T, 15.5 T, 15.6 T, 15.7 T, 15.8 T, 15.9 T, 16.0 T, 16.1 T, 16.2 T, 16.3 T, 16.4 T, 16.5 T, 16.6 T, 16.7 T, 16.8 T, 16.9 T, 17.0 T, 17.1 T, 17.2 T, 17.3 T, 17.4 T, 17.5 T, 17.6 T, 17.7 T, 17.8 T, 17.9 T, 18.0 T, 18.1 T, 18.2 T, 18.3 T, 18.4 T, 18.5 T, 18.6 T, 18.7 T, 18.8 T, 18.9 T, 19.0 T, 19.1 T, 19.2 T, 19.3 T, 19.4 T, 19.5 T, 19.6 T, 19.7 T, 19.8 T, 19.9 T, 20.0 T, 20.1 T, 20.2 T, 20.3 T, 20.4 T, 20.5 T, 20.6 T, 20.7 T, 20.8 T, 20.9 T, or more. Furthermore, the superconducting coils may be used in generating magnetic fields that are outside the range of 2.5 T to 20 T or that are within the range of 3 T to 20 T but that are not specifically listed herein.

By generating a high magnetic field having a magnitude such as those described above, the bend radius of particles orbiting within cavity 84 can be reduced. As a result of the reduction in the bend radius, a greater number of particle orbits can be made within a given-sized cavity. So, the same number of orbits can be fit within a smaller cavity. Reducing the size of the cavity reduces the size of the particle accelerator in general, since a smaller cavity requires smaller magnetic yokes or pole pieces, among other components. In some implementations, the size or volume of the particle accelerator may be 4 m³ or less, 3 m³ or less, or 2 m³ or less.

In some implementations, such as the implementations shown in FIG. 20, the relatively large ferromagnetic magnetic yokes 80, 81 act as magnetic returns for stray magnetic fields produced by the superconducting coils. In some systems, a magnetic shield (not shown) surrounds the yokes. The return yokes and the shield together act to reduce stray magnetic fields, thereby reducing the possibility that stray magnetic fields will adversely affect the operation of the particle accelerator.

In some implementations, the return yokes and/or shield may be replaced by, or augmented by, an active return system. An example active return system includes one or more active return coils that conduct current in a direction opposite to current through the main superconducting coils. In some implementations, there is an active return coil for each superconducting main coil, e.g., two active return coils—one for each main superconducting coil. Each active return coil may also be a superconducting coil that surrounds the outside of a corresponding main superconducting coil concentrically. In some implementations, the active return coils may be or include non-superconducting coils. By using an active return system, the relatively large ferromagnetic magnetic yokes 80, 81 can be replaced with magnetic pole pieces that are smaller and lighter. Accordingly, the size and weight of the synchrocyclotron can be reduced further without sacrificing performance. An example of an active return system that may be used is described in U.S. Pat. No. 8,791,656 (Zwart) entitled “Active Return System”. The content of U.S. Pat. No. 8,791,656, particularly the content related to the return coil configuration (e.g., FIGS. 2, 4, and 5 of U.S. Pat. No. 8,791,656 and the accompanying description), is incorporated herein by reference.

Another example of a particle accelerator that may be used in the particle therapy system herein is described in U.S. Pat. No. 8,975,836 (Bromberg) entitled “Ultra-Light Magnetically Shielded High-Current, Compact Cyclotron”. The content of U.S. Pat. No. 8,975,836, particularly the content related to “cyclotron 11” or “iron-free cyclotron 11” of FIGS. 4, 17 and 18 of U.S. Pat. No. 8,975,836 and the accompanying description, is incorporated herein by reference.

In some implementations, the synchrocyclotron used in the proton therapy system described herein may be a variable-energy synchrocyclotron. In some implementations, a variable-energy synchrocyclotron is configured to vary the energy of the output particle beam by varying the magnetic field in which the particle beam is accelerated. For example, the current may be set to any one of multiple values to produce a corresponding magnetic field. For example, the current may be set to one of two values to produce the dual-energy particle accelerator described previously. In an example implementation, one or more sets of superconducting coils receives variable electrical current to produce a variable magnetic field in the cavity. In some examples, one set of coils receives a fixed electrical current, while one or more other sets of coils receives a variable current so that the total current received by the coil sets varies. In some implementations, all sets of coils are superconducting. In some implementations, some sets of coils, such as the set for the fixed electrical current, are superconducting, while other sets of coils, such as the one or more sets for the variable current, are non-superconducting (e.g., copper) coils.

Generally, in a variable-energy synchrocyclotron, the magnitude of the magnetic field is scalable with the magnitude of the electrical current. Adjusting the total electric current of the coils in a predetermined range can generate a magnetic field that varies in a corresponding, predetermined range. In some examples, a continuous adjustment of the electrical current can lead to a continuous variation of the magnetic field and a continuous variation of the output beam energy. Alternatively, when the electrical current applied to the coils is adjusted in a non-continuous, step-wise manner, the magnetic field and the output beam energy also varies accordingly in a non-continuous (step-wise) manner. The step-wise adjustment can produce the dual energies described previously. In some implementations, each step is between 10 MeV and 80 MeV in size. The scaling of the magnetic field to the current can allow the variation of the beam energy to be carried out relatively precisely, thus reducing the need for an energy degrader. An example of a variable-energy synchrocyclotron that may be used in the particle therapy systems described herein is described in U.S. Pat. No. 9,730,308 entitled “Particle Accelerator That Produces Charged Particles Having Variable Energies”. The content U.S. Pat. No. 9,730,308 is incorporated herein by reference, particularly the content that enables operation of a synchrocyclotron at variable energies, including the content described in columns 5 through 7 of U.S. Pat. No. 9,730,308 and FIG. 13 and its accompanying description.

In implementations of the particle therapy system that use a variable-energy synchrocyclotron, controlling the energy of the particle beam to treat a portion of the irradiation target may be performed in accordance with the treatment plan by changing the energy of the particle beam output by the synchrocyclotron. In such implementations, an energy degrader may or may not be used. For example, controlling the energy of the particle beam may include setting the current in the synchrocyclotron main coils to one of multiple values, each which corresponds to a different energy at which the particle beam is output from the synchrocyclotron. An energy degrader may be used along with a variable-energy synchrocyclotron to provide additional changes in energy, for, example, between discrete energy levels provided by the synchrocyclotron.

The particle therapy system and its variations described herein may be used to apply ultra-high dose rates of radiation—so called, “FLASH” dose rates of radiation—to an irradiation target in a patient. In this regard, experimental results in radiation therapy have shown an improvement in the condition of healthy tissue subjected to radiation when the treatment dose is delivered at ultra-high (FLASH) dose rates. In an example, when delivering doses of radiation at 10 to 20 Gray (Gy) in pulses of less than 500 milliseconds (ms) reaching effective dose rates of 20 to 100 Gray-per-second (Gy/S), healthy tissue experiences less damage than when irradiated with the same dose over a longer time scale, while tumors are treated with similar effectiveness. A theory that may explain this “FLASH effect” is based on the fact that radiation damage to tissue is proportionate to oxygen supply in the tissue. In healthy tissue, the ultra-high dose rate radicalizes the oxygen only once, as opposed to dose applications that radicalize the oxygen multiple times over a longer timescale. This may lead to less damage in the healthy tissue using the ultra-high dose rate.

In some examples, as noted above, ultra-high dose rates of radiation may include doses of radiation that exceed 1 Gray-per-second for a duration of less than 500 ms. In some examples, ultra-high dose rates of radiation may include doses of radiation that exceed 1 Gray-per-second for a duration that is between 10 ms and 5 s. In some examples, ultra-high dose rates of radiation may include doses of radiation that exceed 1 Gray-per-second for a duration that is less than 5 s.

In some examples, ultra-high dose rates of radiation include doses of radiation that exceed one of the following doses for a duration of less than 500 ms: 2 Gray-per-second, 3 Gray-per-second, 4 Gray-per-second, 5 Gray-per-second, 6 Gray-per-second, 7 Gray-per-second, 8 Gray-per-second, 9 Gray-per-second, 10 Gray-per-second, 11 Gray-per-second, 12 Gray-per-second, 13 Gray-per-second, 14 Gray-per-second, 15 Gray-per-second, 16 Gray-per-second, 17 Gray-per-second, 18 Gray-per-50 second, 19 Gray-per-second, 20 Gray-per-second, 30 Gray-per-second, 40 Gray-per-second, 50 Gray-per-second, 60 Gray-per-second, 70 Gray-per-second, 80 Gray-per-second, 90 Gray-per-second, or 100 Gray-per-second. In some examples, ultra-high dose rates of radiation include doses of radiation that exceed one of the following doses for a duration that is between 10 ms and 5 s: 2 Gray-per-second, 3 Gray-per-second, 4 Gray-per-second, 5 Gray-per-second, 6 Gray-per-second, 7 Gray-per-second, 8 Gray-per-second, 9 Gray-per-second, 10 Gray-per-second, 11 Gray-per-second, 12 Gray-per-second, 13 Gray-per-second, 14 Gray-per-second, 15 Gray-per-second, 16 Gray-per-second, 17 Gray-per-second, 18 Gray-per-second, 19 Gray-per-second, 20 Gray-per-second, 30 Gray-per-second, 40 Gray-per-second, 50 Gray-per-second, 60 Gray-per-second, 70 Gray-per-second, 80 Gray-per-second, 90 Gray-per-second, or 100 Gray-per-second. In some examples, ultra-high dose rates of radiation include doses of radiation that exceed one of the following doses for a duration that is less than 5 s: 2 Gray-per-second, 3 Gray-per-second, 4 Gray-per-second, 5 Gray-per-second, 6 Gray-per-second, 7 Gray-per-second, 8 Gray-per-second, 9 Gray-per-second, 10 Gray-per-second, 11 Gray-per-second, 12 Gray-per-second, 13 Gray-per-second, 14 Gray-per-second, 15 Gray-per-second, 16 Gray-per-second, 17 Gray-per-second, 18 Gray-per-second, 19 Gray-per-second, 20 Gray-per-second, 30 Gray-per-second, 40 Gray-per-second, 50 Gray-per-second, 60 Gray-per-second, 70 Gray-per-second, 80 Gray-per-second, 90 Gray-per-second, or 100 Gray-per-second.

In some examples, ultra-high dose rates of radiation include doses of radiation that exceed one or more of the following doses for a duration of less than 500 ms, for a duration that is between 10 ms and 5 s, or for a duration that is less than 5 s: 100 Gray-per-second, 200 Gray-per-second, 300 Gray-per-second, 400 Gray-per-second, or 500 Gray-per-second.

In some examples, ultra-high dose rates of radiation include doses of radiation that are between 20 Gray-per-second and 100 Gray-per-second for a duration of less than 500 ms. In some examples, ultra-high dose rates of radiation include doses of radiation that are between 20 Gray-per-second and 100 Gray-per-second for a duration that is between 10 ms and 5 s. In some examples, ultra-high dose rates of radiation include doses of radiation that are between 20 Gray-per-second and 100 Gray-per-second for a duration that is less than 5 s. In some examples, ultra-high dose rate rates of radiation include doses of radiation that are between 40 Gray-per-second and 120 Gray-per-second for a time period such as less than 5 s. Other examples of the time period are those provided above.

In some implementations, the particle therapy systems may treat three-dimensional columns of the target using ultra-high dose rate radiation—the FLASH doses of radiation. These systems scale the ultra-high dose rate deliveries to targets using pencil beam scanning. In some examples, pencil beam scanning includes delivering a series of small beams of particle radiation that can each have a unique direction, energy, and charge. By combining doses from these individual beams, a three-dimensional target treatment volume may be treated with radiation. Furthermore, instead of organizing the treatment into layers at constant energies, the systems organize the treatment into columns defined by the direction of a stationary beam. The direction of the beam may be toward the surface of the target.

In some implementations, all or part of a column is treated before the particle beam is directed along another path through the irradiation target. In some implementations, a path through the target is all or part-way through the target. In an example, the particle beam may be directed along a path through a target and not deviate from that path. While directed along that path, the energy of the particle beam is changed. The particle beam does not move as its energy changes and, as a result, the particle beam treats all or a part of an interior portion of the target that extends along a length of the particle beam and along a width of the beam spot. The treatment is thus depth-wise along a longitudinal direction of the beam. For example, a portion of the target treated may extend from a spot of the beam at the surface of the target down through all or part of an interior of the target. The result is that the particle beam treats a three-dimensional columnar portion of the target using an ultra-high dose rate of radiation. In some examples, the particle beam may never again be directed along the same three-dimensional columnar portion more than once.

In some implementations, an irradiation target may be broken into micro-volumes. Although cubical micro-volumes may be used, the micro-volumes may have any appropriate shape, such as three-dimensional orthotopes, regular curved shapes, or irregular or amorphous shapes. In this example, each micro-volume is treated through delivery of FLASH radiation by column in the manner described herein. For example, column depths of a micro-volume may be treated with radiation by using energy degrader plates to change the beam energy or by controlling a variable-energy synchrocyclotron to change the beam energy. After an individual micro-volume has been treated, the next micro-volume is treated, and so forth until the entire irradiation target has been treated. Treatment of the micro-volumes may be in any appropriate order or sequence.

In some implementations, a particle accelerator other than a synchrocyclotron may be used in the particle therapy system described herein. For example, a cyclotron, a synchrotron, a linear accelerator, or the like may be substituted for the synchrocyclotron in the particle therapy systems described herein.

One or more scanning magnets (not shown) may be located in the particle beam path between the particle accelerator and the treatment couch. The scanning magnets may be superconducting, non-superconducting, or a combination of superconducting and non-superconducting. The scanning magnets may be of the type shown in FIG. 5, in FIGS. 7, 8, 11, 12A, 12B, 23 or a combination thereof, for example. Control over scanning is achieved, in some implementations, by varying current through one or both sets of coils to thereby vary the magnetic field(s) produced thereby. By varying the magnetic field(s) appropriately, the particle beam can be moved in the X and/or Y dimension across the irradiation target.

In some implementations, the scanning magnet(s) may be replaced with a scattering foil and the energy degrader may be a range modulator. In implementations such as this, the scattering foil scatters the particle beam across a treatment area and the depth to which the scattered beam is applied is controlled by the range modulator. The configurable collimator may remain in place to trim edges of the scattered beam.

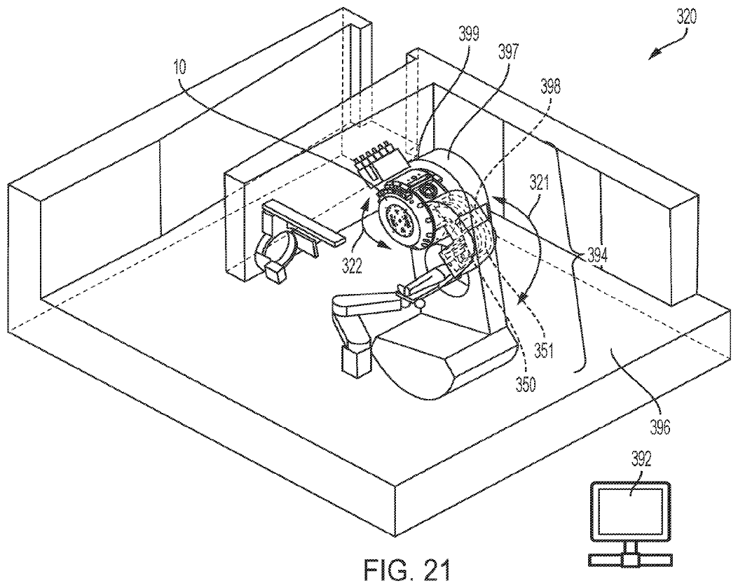

Another example particle therapy system 320 that uses the bending magnets described herein is shown in FIG. 21. In FIG. 21, gantry 394 may be rotationally or axially connected to a treatment room floor 396, enabling controlled movement of gantry 394 relative to the treatment room floor. In this example, particle accelerator 10 is mounted on the gantry and is rotatable in the directions of arrows 321 around the patient with the gantry to direct the particle beam toward the patient. Gantry 394 may include an arm 397 that runs the length of gantry 394 and that reaches the treatment room floor 396. Particle accelerator 10 and connected beamline structure 398 are rotatably mounted to arm 397. That is, particle accelerator 10 and connected beamline structure 398 are connected to an end 399 of arm 397 so that particle accelerator 10 and connected beamline structure 398 are able to rotate at end 399 in the directions of arrows 322. This rotation is separate from the gantry rotation described herein. The beamline structure 398 may contain one or more bending magnets of the type described with respect to FIGS. 25 to 31 or any variant thereof. For example, the beamline structure may include two bending magnets 350 and 351 of the type described with respect to FIGS. 25 to 31 or any variant thereof to bend the particle beam by more than 90° towards the irradiation target, such as 100°, 110°, 120°, or more.

Operation of the example proton therapy systems described herein, and operation of all or some component thereof, can be controlled, at least in part, using a control system 192 (FIG. 1) or 392 (FIG. 21) configured to execute one or more computer program products, e.g., one or more computer programs tangibly embodied in one or more non-transitory machine-readable media, for execution by, or to control the operation of, one or more data processing apparatus, e.g., a programmable processor, a computer, multiple computers, and/or programmable logic components.

All or part of the systems described in this specification and their various modifications may be configured or controlled at least in part by one or more computers such as the control system using one or more computer programs tangibly embodied in one or more information carriers, such as in one or more non-transitory machine-readable storage media. A computer program can be written in any form of programming language, including compiled or interpreted languages, and it can be deployed in any form, including as a stand-alone program or as a module, part, subroutine, or other unit suitable for use in a computing environment. A computer program can be deployed to be executed on one computer or on multiple computers at one site or distributed across multiple sites and interconnected by a network.

Actions associated with configuring or controlling the systems described herein can be performed by one or more programmable processors executing one or more computer programs to control or to perform all or some of the operations described herein. All or part of the systems and processes can be configured or controlled by special purpose logic circuitry, such as, an FPGA (field programmable gate array) and/or an ASIC (application-specified integrated circuit) or embedded microprocessor(s) localized to the instrument hardware.

Processors suitable for the execution of a computer program include, by way of example, both general and special purpose microprocessors, and any one or more processors of any kind of digital computer. Generally, a processor will receive instructions and data from a read-only storage area or a random access storage area or both. Elements of a computer include one or more processors for executing instructions and one or more storage area devices for storing instructions and data. Generally, a computer will also include, or be operatively coupled to receive data from, or transfer data to, or both, one or more machine-readable storage media, such as mass storage devices for storing data, such as magnetic, magneto-optical disks, or optical disks. Non-transitory machine-readable storage media suitable for embodying computer program instructions and data include all forms of non-volatile storage area, including by way of example, semiconductor storage area devices, such as EPROM (erasable programmable read-only memory), EEPROM (electrically erasable programmable read-only memory), and flash storage area devices; magnetic disks, such as internal hard disks or removable disks; magneto-optical disks; and CD-ROM (compact disc read-only memory) and DVD-ROM (digital versatile disc read-only memory).

Elements of different implementations described may be combined to form other implementations not specifically set forth previously. Elements may be left out of the systems described previously without adversely affecting their operation or the operation of the system in general. Furthermore, various separate elements may be combined into one or more individual elements to perform the functions described in this specification.

Other implementations not specifically described in this specification are also within the scope of the following claims.

What is claimed is:

1. A magnet comprising:
an assembly comprising: (i) sets of coils for conducting current to produce a magnetic field, and (ii) a support structure on which the sets of coils are disposed asymmetrically; and
a ferromagnetic yoke surrounding part of the assembly, the ferromagnetic yoke and the assembly being bent.

2. The magnet of claim 1, wherein the sets of coils comprise a first coil and a second coil, the first coil and the second coil for conducting current to produce a magnetic field, the first coil and the second coil being disposed on the support structure asymmetrically in a first hemisphere of the magnet such that a first spacing between the first coil and the second coil in a first quadrant of the magnet is different from a second spacing between the first coil and the second coil in a second quadrant of the magnet, the first quadrant and the second quadrant being within the first hemisphere;
wherein the sets of coils comprise a third coil and a fourth coil, the third coil and the fourth coil for conducting current to produce a magnetic field, the third coil and the fourth coil being disposed on the support structure asymmetrically in a second hemisphere of the magnet such that a third spacing between the third coil and the fourth coil in a third quadrant of the magnet is different from a fourth spacing between the third coil and the fourth coil in a fourth quadrant of the magnet, the third quadrant and the fourth quadrant being within the second hemisphere; and
wherein an asymmetry of the first and second coils in the first and second quadrants, respectively, mirrors an asymmetry of the third and fourth coils in the third and fourth quadrants, respectively.

3. The magnet of claim 2, wherein the first spacing and the third spacing are equal, the second spacing and the fourth spacing are equal, and the first spacing and the third spacing are less than the second spacing and the fourth spacing; and
wherein the first spacing and the third spacing are at an inner bend radius of the assembly and the second spacing and the fourth spacing are at an outer bend radius of the assembly.

4. The magnet of claim 3, wherein the sets of coils comprise a fifth coil and a sixth coil, the fifth coil and the sixth coil for conducting current to produce a magnetic field, the fifth coil being disposed on the support structure in the first hemisphere and the sixth coil being disposed on the support structure in the second hemisphere;
wherein a fifth spacing between the fifth coil and an adjacent one of the first or second coils in the first quadrant is different than a sixth spacing between the fifth coil and an adjacent one of the first or second coils in the second quadrant;
wherein a seventh spacing between the sixth coil and an adjacent one of the third or fourth coils in the third quadrant is different than an eighth spacing between the sixth coil and an adjacent one of the third or fourth coils in the fourth quadrant; and
wherein an asymmetry of the first, second, and fifth coils in the first and second quadrants, respectively, mirrors an asymmetry of the third, fourth, and sixth coils in the third and fourth quadrants, respectively.

5. The magnet of claim 4, wherein the fifth spacing and the seventh spacing are equal, the sixth spacing and the eighth spacing are equal, and the fifth spacing and the seventh spacing are less than the sixth spacing and the eighth spacing; and
wherein the fifth spacing and the seventh spacing are at the inner bend radius of the assembly and the sixth spacing and the eighth spacing are at the outer bend radius of the assembly.

6. The magnet of claim 5, wherein the sets of coils comprise a seventh coil and an eighth coil, the seventh coil and the eighth coil for conducting current to produce a magnetic field, the seventh coil being disposed on the support structure in the first hemisphere and the eighth coil being disposed on the support structure in the second hemisphere;
wherein a ninth spacing between the seventh coil and an adjacent one of the first, second, or fifth coils in the first quadrant is different than a tenth spacing between the seventh coil and an adjacent one of the first, second, or fifth coils in the second quadrant;
wherein an eleventh spacing between the eighth coil and an adjacent one of the third, fourth, or sixth coils in the third quadrant is different than a twelfth spacing between the eighth coil and an adjacent one of the third, fourth, or sixth coils in the fourth quadrant; and
wherein an asymmetry of the first, second, fifth, and seventh coils in the first and second quadrants, respectively, mirrors an asymmetry of the third, fourth, sixth, and eighth coils in the third and fourth quadrants, respectively.

7. The magnet of claim 6, wherein the ninth spacing and the eleventh spacing are equal, the tenth spacing and the twelfth spacing are equal, and the ninth spacing and the eleventh spacing are less than the tenth spacing and the twelfth spacing; and
wherein the ninth spacing and the eleventh spacing are at the inner bend radius of the assembly and the tenth spacing and the twelfth spacing are at the outer bend radius of the assembly.

8. The magnet of claim 2, wherein the ferromagnetic yoke comprises notches adjacent to the assembly, the notches being asymmetric in the first quadrant and the second quadrant, where an asymmetry of the notches is with respect to at least one of a size, shape, or placing of the notches; and
wherein an asymmetry of the notches in the third and fourth quadrants, respectively, mirrors an asymmetry of the notches in the first and second quadrants, respectively.

9.-10. (canceled)

11. The magnet of claim 1, wherein the ferromagnetic yoke comprises iron; and
wherein the support structure is non-ferromagnetic.

12. The magnet of claim 1, wherein the magnet is bent by 60° or more relative to a straight line passing through a center of an unbent part of the magnet.

13.-16. (canceled)

17. The magnet of claim 1, wherein the magnet is a cosine-theta magnet in which current through the sets coils has a greater concentration near a 0° or 180° location of the magnet than near a 90° or −90/270° location of the magnet.

18. The magnet of claim 1, wherein the sets of coils are configured for dipole functionality; or
wherein the sets of coils are configured for quadrupole functionality; or
wherein the sets of coils are configured for sextupole functionality.

19.-20. (canceled)

21. The magnet of claim 1, wherein the sets of coils comprise superconducting material.

22. The magnet of claim 1, further comprising:
one or more magnetic shims that are movable relative to the magnetic yoke to change a magnetic field produced by the magnet.

23. The magnet of claim 1, wherein the sets of coils comprise two or more sets of coils that are configured asymmetrically relative to a first dimension and symmetrically relative to a second dimension, the first dimension being perpendicular to the second dimension.

24. A system comprising:
a gantry comprising a beamline structure configured to direct a particle beam that is monoenergetic from an output of a particle accelerator towards an irradiation target, the beamline structure comprising bending magnets to bend the particle beam along a length of the beamline structure;
wherein at least one of the bending magnets comprises the magnet of claim 1.

25.-26. (canceled)

27. The system of claim 24, wherein the at least one bending magnet comprises a magnet having a magnetic field of 2.5 Tesla (T) or more; or wherein the at least one bending magnet comprises a magnet having a magnetic field of 3 Tesla (T) or more.

28.-29. (canceled)

30. The system of claim 24, wherein the gantry comprises a support structure configured to move part of the beamline structure in a circular path around the irradiation target; and
wherein the support structure has a dimension that is 6 meters or less.

31.-36. (canceled)

37. The system of claim 24, wherein the beamline structure comprises an output channel comprising at least some of the bending magnets, the at least one bending magnet preceding the output channel in a direction of travel of the particle beam.

38. The system of claim 24, wherein the gantry is an achromat from an entry point of a particle beam into the gantry to an isocenter of the system.
